(19) United States
(12) Patent Application Publication
(10) Pub. No.: US 2010/0086981 A1
(43) Pub. Date: Apr. 8, 2010

(54) COMPOSITIONS AND METHODS FOR IMPROVED SACCHARIFICATION OF BIOMASS

(75) Inventors: William G. LaTouf, Ashland, MA (US); Matthias Schmalisch, Marlborough, MA (US); Kexue Huang, Westborough, MA (US); Muhammad Abdul Ali, Marlborough, MA (US)

Correspondence Address: WILSON, SONSINI, GOODRICH & ROSATI, 650 PAGE MILL ROAD, PALO ALTO, CA, 94304-1050, US

(73) Assignee: QTEROS, INC., Marlborough, MA (US)

(21) Appl. No.: 12/630,784

(22) Filed: Dec. 3, 2009

Related U.S. Application Data
(60) Provisional application No. 61/221,523, filed on Jun. 29, 2009.

(51) Int. Cl. C12P 7/56; C12P 1/00; C12P 7/40; C12P 7/54; C12P 7/06; C12P 7/08; C12P 1/04

(52) U.S. Cl. 435/139; 435/41; 435/136; 435/140; 435/161; 435/163; 435/170

(57) ABSTRACT

Compositions and methods are provided for enhancing saccharification of biomass with one or more enzymes to enhance availability of substrates for fermentation by a microorganism. Microorganisms are also modified to enhance activity of one or more hydrolytic enzymes that are present endogenously in or are introduced heterologously into a host microorganism.

### CLAIM OF PRIORITY

This application claims the benefit of priority from U.S. Provisional Patent Application Ser. No. 61/221,523, filed on Jun. 29, 2009, the entire contents of which are incorporated by reference herein.

### SEQUENCE LISTING

The instant application contains a Sequence Listing which has been submitted via EFS-Web and is hereby incorporated by reference in its entirety. Said ASCII copy, created on Nov. 30, 2009, is named 37836717.txt, and is 81,443 bytes in size.

### BACKGROUND OF THE INVENTION

Biomass is a renewable source of energy, which can be biologically fermented to produce an end-product such as a fuel (e.g. alcohol, ethanol, organic acid, acetic acid, lactic acid, methane, or hydrogen). Biomass includes agricultural residues (corn stalks, grass, straw, grain hulls, bagasse, etc.), animal waste (manure from cattle, poultry, and hogs), woody materials (wood or bark, sawdust, timber slash, and mill scrap), municipal waste (waste paper, recycled toilet papers, yard clippings, etc.), and energy crops (poplars, willows, switch grass, alfalfa, prairie bluestem, algae etc.). Lignocellulosic biomass has cellulose and hemicellulose as two major components. To obtain a high fermentation efficiency of lignocellulosic biomass to end-product (yield) it is important to provide an appropriate fermentation environment to enhance end-product yield. More complete saccharification of biomass and fermentation of the saccharification products results in higher fuel yields.

Unfortunately, many organisms used for fermentation of carbonaceous substrates cannot saccharify polysaccharides into monosaccharides. Progress in bioproduct fermentation has been hampered by lack of suitable microorganisms that can effectively hydrolyze and metabolize all of the sugars present in a biomass. This increases the cost of fermentation because hydrolysis must be accomplished by the addition of expensive mixtures of enzymes that can hydrolyze five and six carbon polysaccharides. There is considerable need for organisms that can efficiently utilize polysaccharides such as cellulose and hemicellulose with little or no enzyme addition during fermentation.

### SUMMARY OF THE INVENTION

In one aspect of the invention, a process is provided for producing a fermentive end-product comprising contacting a carbonaceous biomass with (a) a microorganism that hydrolyses and ferments said biomass; and (b) an external source of one or more enzymes that are capable of enhancing said hydrolysis, and allowing sufficient time for said hydrolysis and fermentation to produce a fermentive end-product wherein the one or more enzymes do not include a xylanase, a hemicellulase, a glucanase or glucosidase, and wherein said external source is not the microorganism.

In another aspect of the invention, a process is provided for producing a fermentive end-product comprising contacting a carbonaceous biomass with (a) a microorganism that hydrolyses and ferments said biomass; and (b) an external source of a single enzyme that is capable of enhancing said hydrolysis, and allowing sufficient time for the hydrolysis and fermentation to produce a fermentive end-product wherein the enzyme is a cellulase and wherein the external source is not the microorganism.

In a further aspect of the invention, a process is provided for producing a fermentive end-product comprising contacting a carbonaceous biomass with (a) a microorganism that hydrolyses and ferments the biomass; and (b) an external source of one or more enzymes that are capable of enhancing said hydrolysis, and allowing sufficient time for the hydrolysis and fermentation to produce a fermentive end-product wherein the one or more enzymes comprise cellulase, and wherein the cellulase and the microorganism act synergistically to enhance hydrolysis.

In another aspect of the invention, a process is provided for producing a fermentive end-product comprising contacting a carbonaceous biomass with (a) a recombinant microorganism that hydrolyses and ferments the biomass; and (b) an external source of one or more enzymes that are capable of enhancing the hydrolysis, and allowing sufficient time for the hydrolysis and fermentation to produce a fermentive end-product.

Any of the above processes can be carried out where contact is in a large-scale fermentation vessel, wherein the fermentation vessel is adapted to provide suitable conditions for fermentation of one or more carbohydrate into fermentive end-products. Further, any of the above processes can be carried out where the microorganism is capable of direct fermentation of C5 and C6 carbohydrates.

Useful microorganisms include a bacterium, a species of Clostridia, or Clostridium phytofermentans. Further the microorganism can be non-recombinant or recombinant. If recombinant, the microorganism can comprise one or more heterologous polynucleotides that enhance the activity of one or more cellulases, or one or more polynucleotides that encode one or more copies of an endogenous cellulase.

In a further embodiment, in any of the above processes, the biomass can comprise one or more of corn steep solids, corn steep liquor, malt syrup, xylan, cellulose, hemicellulose, fructose, glucose, mannose, rhamnose, or xylose. In another embodiment, in any of the above processes, the biomass can comprise municipal waste, wood, plant material, plant material extract, a natural or synthetic polymer, or a combination thereof. Further, the plant material can be switchgrass, bagasse, corn stover or poplar.

In another embodiment, the fermentive end-product of any of the above processes is an alcohol. Further, the fermentive end-product of any of the above processes can be ethanol, lactic acid, acetic acid or formic acid.

Any of the above processes can also be carried out where the microorganism is capable of uptake of one or more complex carbohydrates. These processes also include biomass that comprises a higher concentration of oligomeric carbohydrates relative to monomeric carbohydrates.

In a further aspect of this invention, the polynucleotides used in the processes of this invention comprise all or a portion of SEQ ID NO: 1, SEQ ID NO: 2, SEQ ID NO: 5, SEQ ID NO: 8, SEQ ID NO: 11, SEQ ID NO: 14, SEQ ID NO: 18, or SEQ ID NO: 32, or one or more polynucleotides that encodes one or more enzymes selected from the group consisting of Cphy_{—}3202, Cphy_{—}2058, Cphy_{—}1163, Cphy_{—}3367, Cphy_{—}1100, Cphy_{—}1510, Cphy_{—}3368, and Cphy_{—}2128.

The processes of this invention also include embodiments wherein the external source of one or more enzymes is in an amount from about 0.4 to about 15 filter paper unit (FPU)/gram cellulose, or wherein cellulase is in an amount from about 0.4 to about 15 filter paper unit (FPU)/gram cellulose, or wherein the enhanced hydrolytic activity is equivalent to addition of cellulases in an amount sufficient to provide activity of about 0.4 to about 15 filter paper unit (FPU)/gram cellulose.

In a further embodiment, the processes of this invention provide hydrolysis that result in a greater concentration of cellobiose and/or larger oligomers, relative to monomeric carbohydrates. The monomeric carbohydrates can comprise xylose and arabinose.

In a further embodiment, the processes of this invention can be carried out with a biomass that is pre-conditioned with alkali treatment.

In another aspect of this invention, the recombinant microorganism useful for hydrolysis and fermentation during the processes of this invention produces one or more hydrolytic enzyme encoded by a variant having a polynucleotide sequence with an identity of 70% or more compared to a sequence selected from SEQ ID NO 1, SEQ ID NO: 2, SEQ ID NO: 5, SEQ ID NO: 8, SEQ ID NO: 11, SEQ ID NO: 14, or a combination thereof.

In a further embodiment of this invention, a product for production of a fermentive end-product can comprise (a) a carbonaceous biomass, (b) a microorganism that hydrolyses and ferments the biomass; and (c) an external source of one or more enzymes that are capable of enhancing the hydrolysis, wherein the one or more enzymes do not include a xylanase, a hemicellulase, a glucanase or glucosidase, and wherein the external source is not said microorganism.

In another aspect of the invention, a product is provided for production of a fermentive end-product, the product comprising (a) a carbonaceous biomass, (b) a microorganism that hydrolyses and ferments the biomass; and (c) an external source of a single enzyme that is capable of enhancing the hydrolysis, wherein one or more enzymes is a cellulase.

In another aspect of the invention, a product is provided for production of a fermentive end-product, the product comprising (a) a carbonaceous biomass, (b) a microorganism that hydrolyses and ferments the biomass; and (c) an external source of one or more enzymes that are capable of enhancing the hydrolysis, wherein the one or more enzymes comprise cellulase, and wherein the cellulase and the microorganism act synergistically to produce an end product.

In another aspect of the invention, a product is provided for production of a fermentive end-product, the product comprising (a) a carbonaceous biomass, (b) a microorganism that hydrolyses and ferments the biomass; and (c) an external source of one or more enzymes that are capable of enhancing the hydrolysis.

This invention further comprises any of the above products wherein the microorganism is capable of direct fermentation of C5 and C6 carbohydrates. A useful microorganism to produce the products of this invention is a bacterium, a species of Clostridia, or Clostridium phytofermentans. The microorganism can be non-recombinant. Further the microorganism can comprise one or more heterologous polynucleotides that enhance the activity of one or more cellulases or can be modified to provide enhanced activity of one or more cellulase.

This invention further includes any of the above products wherein the biomass comprises one or more of corn steep solids, corn steep liquor, malt syrup, xylan, cellulose, hemicellulose, fructose, glucose, mannose, rhamnose, or xylose. The biomass of this invention can also comprise municipal waste, wood, plant material, plant material extract, a natural or synthetic polymer, or a combination thereof. Useful plant material is switchgrass, bagasse, corn stover or poplar.

This invention further comprises any of the above products wherein the fermentive end-product is an alcohol or comprises ethanol, lactic acid, acetic acid and formic acid.

In another embodiment of this invention, the products include a microorganism capable of uptake of one or more complex carbohydrates.

In another embodiment of this invention, the products include a biomass comprising a higher concentration of oligomeric carbohydrates relative to monomeric carbohydrates.

In another embodiment of this invention, the products for production of a fermentive end-product include an enzyme identified in Table 2. In a further embodiment, the products include one or more polynucleotides that are SEQ ID NO: 1, SEQ ID NO: 2, SEQ ID NO: 5, SEQ ID NO: 8, SEQ ID NO: 11, SEQ ID NO: 14, SEQ ID NO: 17, or SEQ ID NO: 32 or one or more polynucleotides encoding Cphy_{—}3202, Cphy_{—}2058, Cphy_{—}1163, Cphy_{—}3367, Cphy_{—}1100, Cphy_{—}1510, Cphy_{—}3368, or Cphy_{—}2128.

One aspect of this invention includes a vector comprising: (a) one or more polynucleotides encoding a biomass degrading enzyme; and (b) a sequence encoding an erythromycin resistance gene. In another embodiment, this invention includes a vector comprising: (a) one or more polynucleotides encoding a biomass degrading enzyme; and (b) a SacII restriction site. Either of these vectors can further comprise a constitutively active promoter. In a further embodiment, the constitutively active promoter is a Cphy_{—}3510 promoter.

The vectors of this invention can further comprise a polynucleotide that encodes Cphy_{—}3289 or Cphy_{—}3290. The vectors of this invention can also include one or more polynucleotides such as SEQ ID NO: 1, SEQ ID NO: 2, SEQ ID NO: 5, SEQ ID NO: 8, SEQ ID NO: 11, SEQ ID NO: 14, SEQ ID NO: 17, or SEQ ID NO: 32 or one or more polynucleotides that hybridizes to SEQ ID NO: 2, SEQ ID NO: 5, SEQ ID NO: 8, SEQ ID NO: 11, SEQ ID NO: 14, SEQ ID NO: 18 or SEQ ID NO: 32 under conditions of medium stringency.

Vectors of this invention can encode biomass degrading enzymes such as Cphy_{—}3202, Cphy_{—}2058, Cphy_{—}1163, Cphy_{—}3367, Cphy_{—}1100, Cphy_{—}1510, Cphy_{—}3368, or Cphy_{—}2128.

In another embodiment, vectors of this invention can comprise polynucleotides encoding a polypeptide of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29.

In another aspect of this invention, vectors can comprise a polynucleotide that encodes a polypeptide that is expressed in a species of gram⁺ bacteria, gram⁻ bacteria, a yeast, or other microbe. The polynucleotide can also encode a cellulase.

In another embodiment, vectors of this invention can encode a biomass degrading enzyme that is a cellulolytic, hemicellulolytic, or ligninolytic enzyme.

A further embodiment of this invention includes a microorganism comprising any of the vectors of the invention. The recombinant microorganism can have increased expression of one or more biomass degrading enzymes as compared to a non-recombinant microorganism, wherein the one or more biomass degrading enzymes are cellulolytic, hemicellulolytic, or ligninolytic enzymes.

Such useful recombinant microorganisms with enhanced hydrolytic activity will express one or more biomass degrading enzymes encoded by SEQ ID NO: 2, SEQ ID NO: 5, SEQ ID NO: 8, SEQ ID NO: 11, or SEQ ID NO: 14, or the chaperonin proteins encoded by SEQ ID NO: 17. Other useful recombinant microorganisms with enhanced hydrolytic activity will express one or more biomass degrading enzymes wherein the one or more biomass degrading enzymes are SEQ ID NO: 20, SEQ ID NO: 21, SEQ ID NO: 22, SEQ ID NO: 23, SEQ ID NO: 24, SEQ ID NO: 25, or express the chaperonin proteins SEQ ID NO: 26 and SEQ ID NO: 27. In a further embodiment, the recombinant microorganisms will express one or more biomass degrading enzymes wherein the one or more biomass degrading enzymes are Cphy_{—}3202, Cphy_{—}2058, Cphy_{—}1163, Cphy_{—}3367, Cphy_{—}1100, Cphy_{—}1510, Cphy_{—}3368, or Cphy_{—}2128. The recombinant microorganism can further comprise one or both of the chaperonin proteins encoded by all or a portion of SEQ ID NO: 17.

The recombinant microorganisms of this invention can comprise a Cphy_{—}3510 promoter.

The recombinant microorganisms of this invention can be species of Clostridium, including but not limited to C. phytofermentans, but they can also be a bacterium or yeast or other fungal cell. The plasmids and promoters of this invention are designed to work in any microbe.

In a further embodiment, the microorganism of this invention can demonstrate enhanced hydrolytic activity that is equivalent to the addition of cellulases in an amount sufficient to provide activity of about 0.4 to about 15 filter paper unit (FPU)/gram cellulose. These microorganisms can comprise one or more heterologous polynucleotides that enhance the activity of one or more cellulases or any biomass-degrading enzyme.

In another aspect of this invention the microorganisms can hydrolyze and ferment biomass that comprises one or more of corn steep solids, corn steep liquor, malt syrup, xylan, cellulose, hemicellulose, fructose, glucose, mannose, rhamnose, or xylose. Further, the biomass can comprise municipal waste, wood, plant material, plant material extract, a natural or synthetic polymer, or a combination thereof.

In another embodiment, the microorganisms of this invention are capable of uptake of one or more complex carbohydrates and can utilize biomass comprising a higher concentration of oligomeric carbohydrates relative to monomeric carbohydrates. In some of these microorganisms, the biomass degrading enzyme is Cphy_{—}3202, Cphy_{—}2058, Cphy_{—}1163, Cphy_{—}3367, Cphy_{—}1100, Cphy_{—}1510, Cphy_{—}3368, or Cphy_{—}2128.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1B illustrates ethanol production from corn stover utilizing a cocktail mix (A) and individual enzyme(s) components of the cocktail mix (B).

FIG. 2 illustrates saccharification yield.

FIG. 3 illustrates ethanol production from corn stover utilizing a cocktail mix (A) and individual enzyme(s) components of the cocktail mix (B).

FIG. 4 illustrates a hydrolysis plot showing C. phytofermentans (Q) saccharification.

FIG. 5 illustrates enzyme-assisted fermentation of corn stover.

FIG. 6 illustrates a pathway map for cellulose hydrolysis and fermentation.

FIG. 7 illustrates a plasmid map for pIMP1.

FIG. 8 illustrates a plasmid map for pIMCphy.

FIG. 9 illustrates a plasmid map for pCphyP3510.

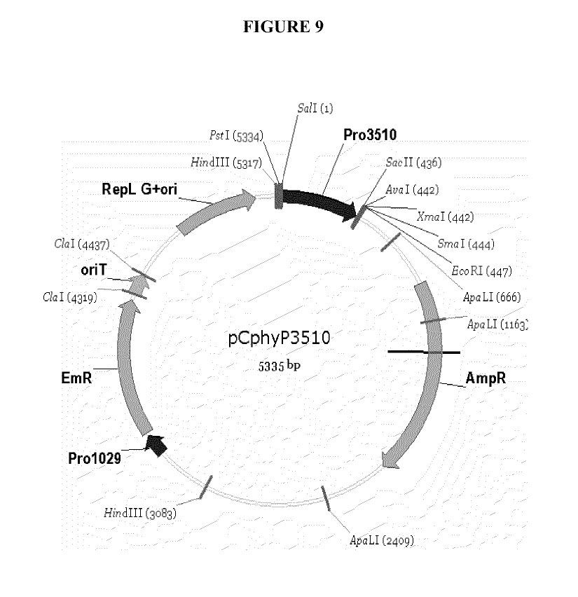

FIG. 10 illustrates CMC-congo red plate and Cellazyme Y assays.

FIG. 11 illustrates a plasmid map for pCphyP3510-1163.

FIG. 12 illustrates the nucleic acid sequence of Cphy_{—}1163 and relevant primers.

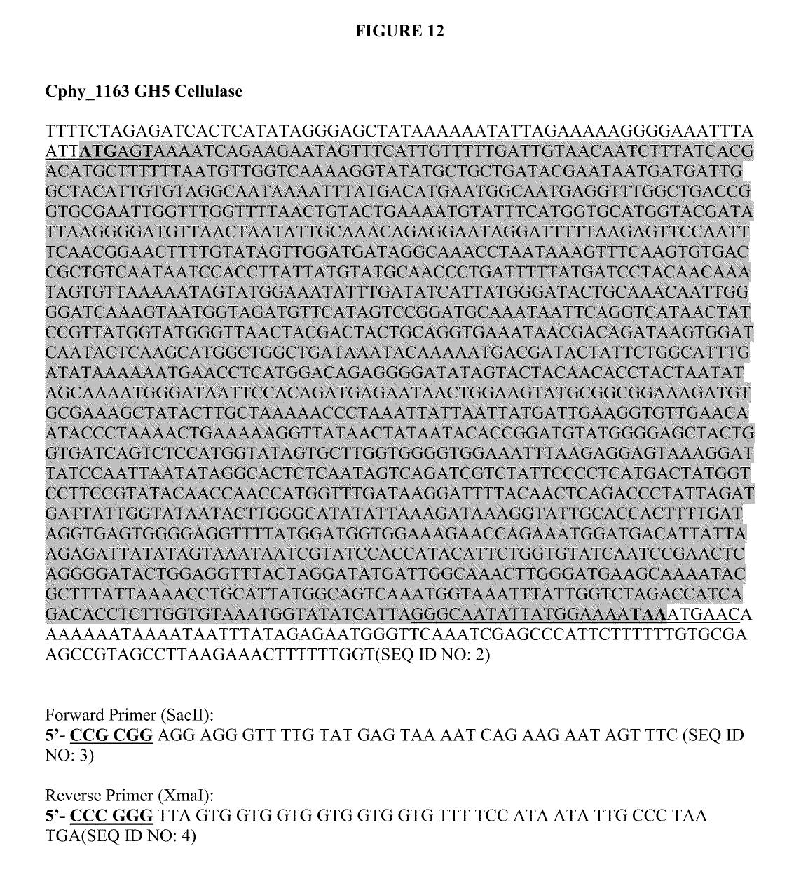

FIG. 13 illustrates the nucleic acid sequence of Cphy_{—}3367 and relevant primers.

FIG. 14 illustrates the nucleic acid sequence of Cphy_{—}3368 and relevant primers.

FIG. 15 illustrates the nucleic acid sequence of Cphy_{—}3202 and relevant primers.

FIG. 16 depicts the nucleic acid sequence of Cphy_{—}2058 and relevant primers.

FIG. 17 depicts the nucleic acid sequences of Cphy_{—}3289 and Cphy_{—}3290 and relevant primers.

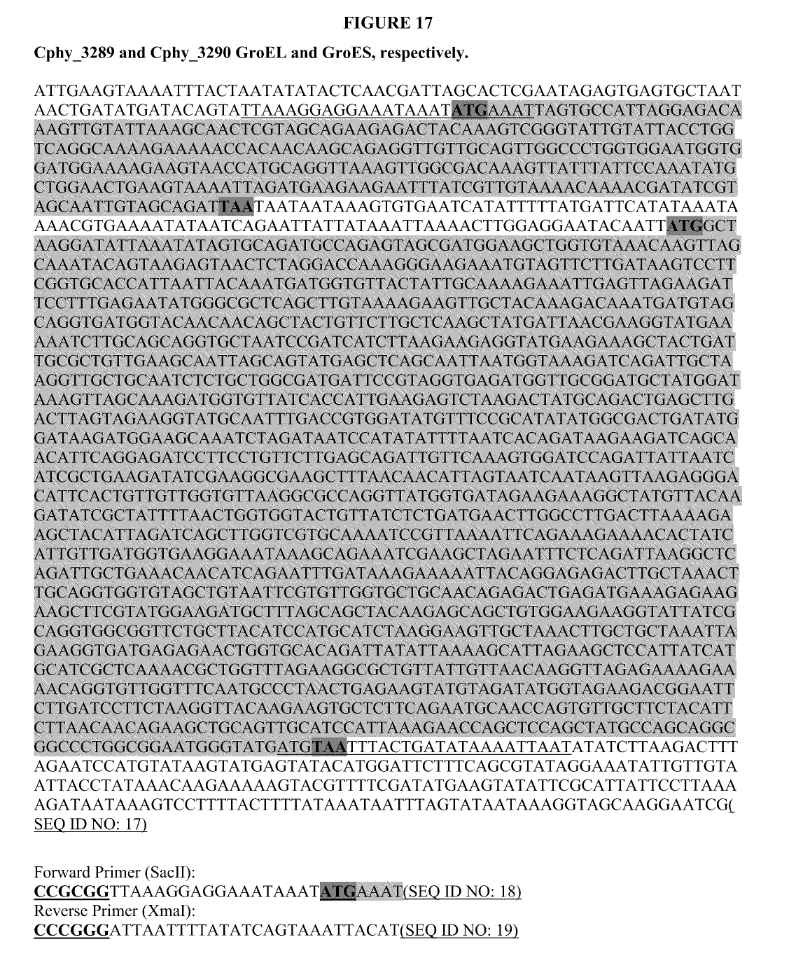

FIG. 18 depicts the amino acid sequence of Cphy_{—}1163.

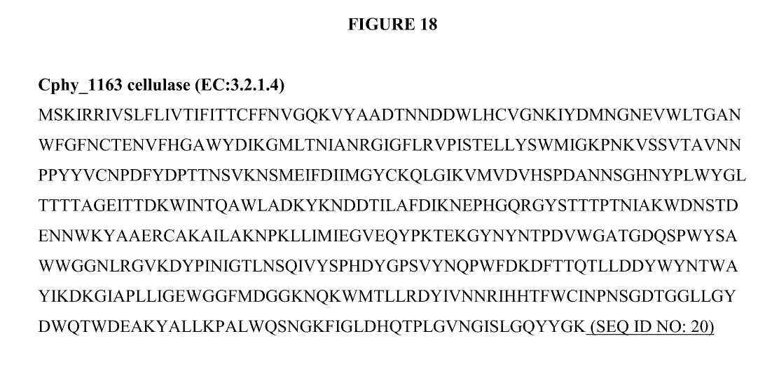

FIG. 19 illustrates the amino acid sequence of Cphy_{—}3367.

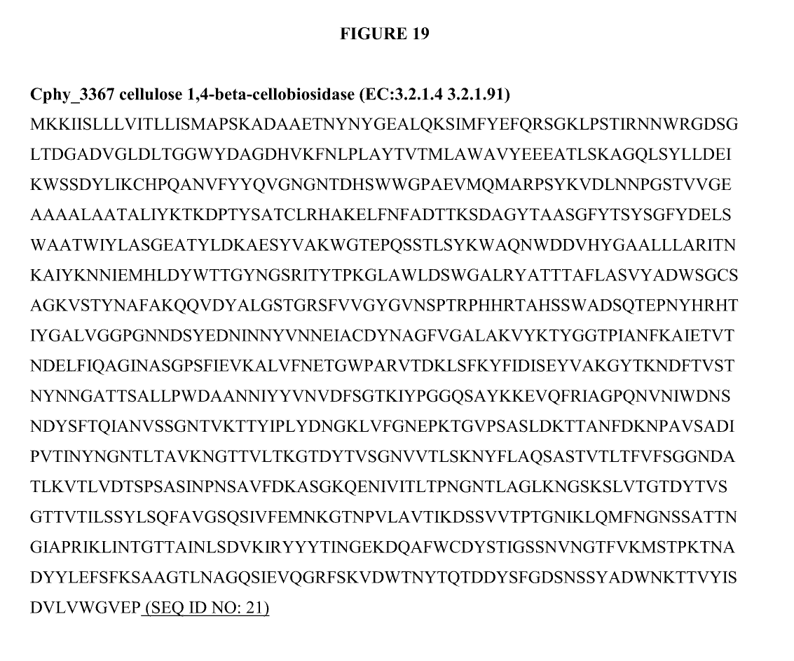

FIG. 20 illustrates the amino acid sequence of Cphy_{—}3368.

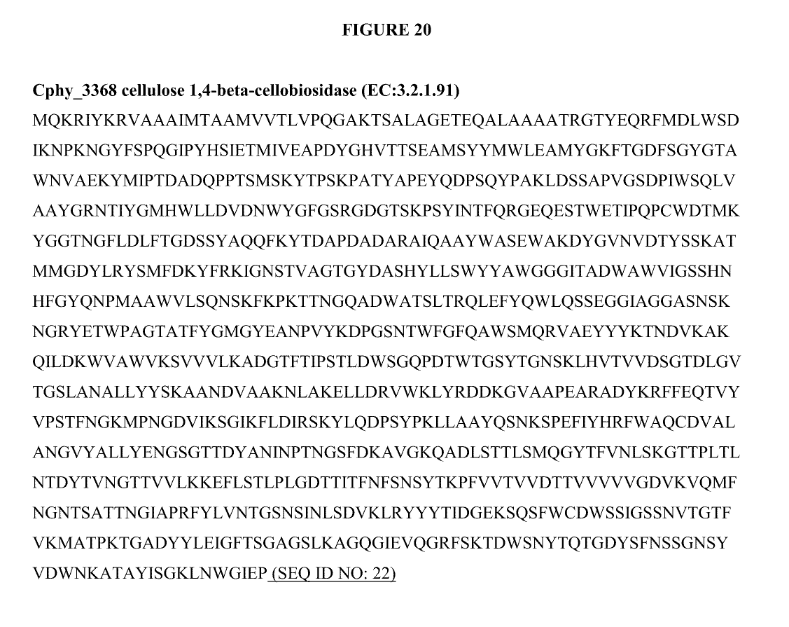

FIG. 21 illustrates the amino acid sequence of Cphy_{—}3202.

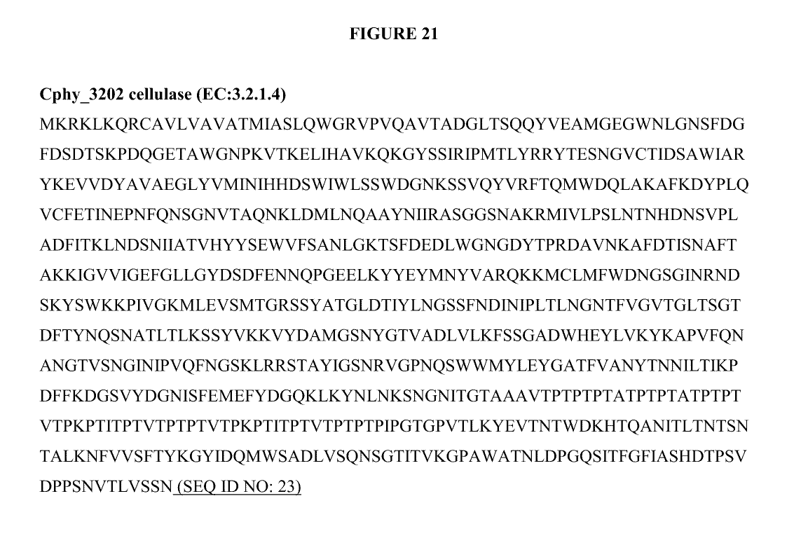

FIG. 22 illustrates the amino acid sequence of Cphy_{—}2058.

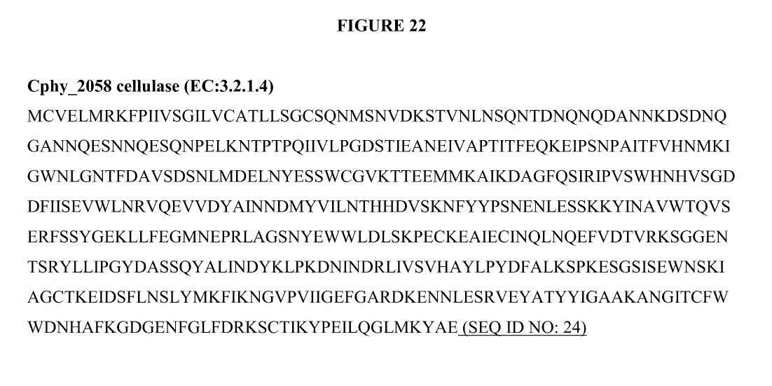

FIG. 23 illustrates the amino acid sequence of Cphy_{—}1100.

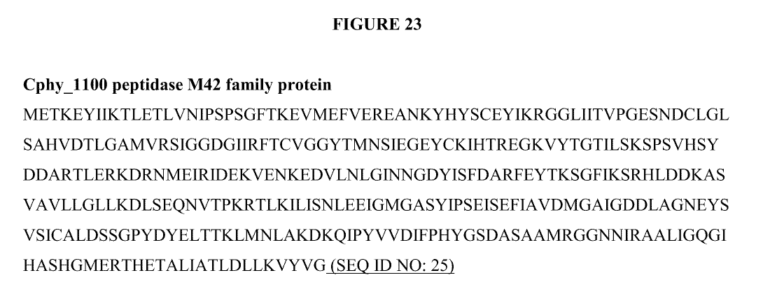

FIG. 24 illustrates the amino acid sequence of Cphy_{—}1510.

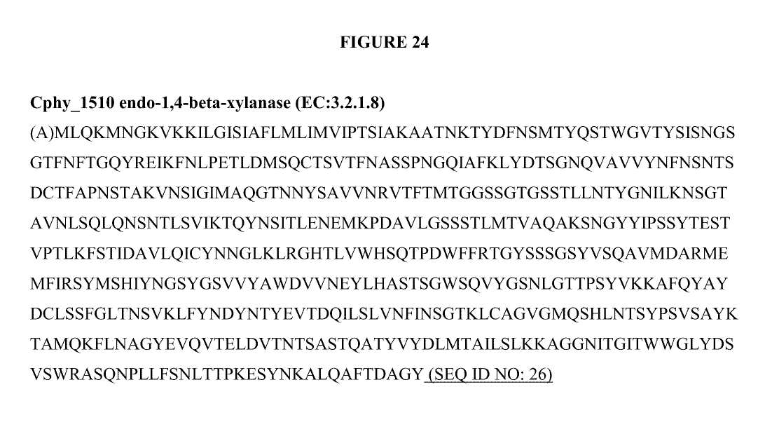

FIG. 25 illustrates the amino acid sequence of Cphy_{—}2128.

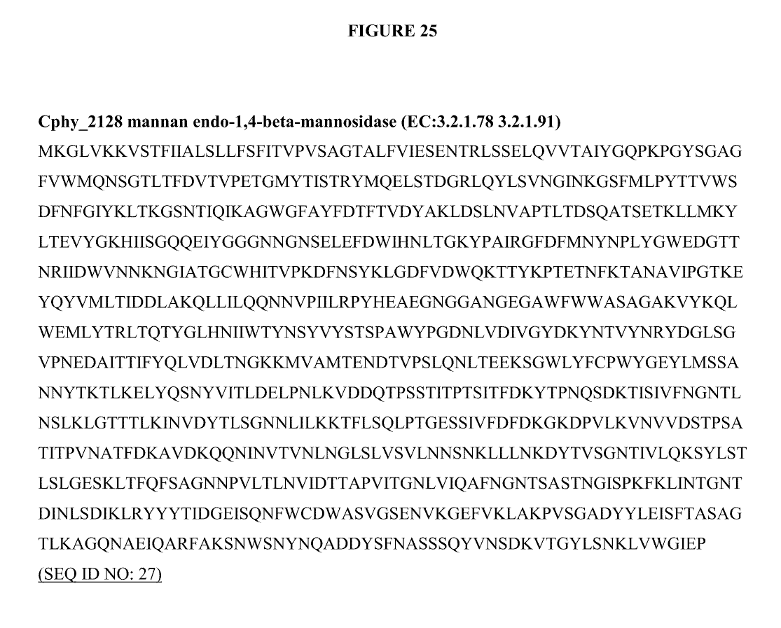

FIG. 26 illustrates the amino acid sequence of Cphy_{—}3289 chaperonin GroEL.

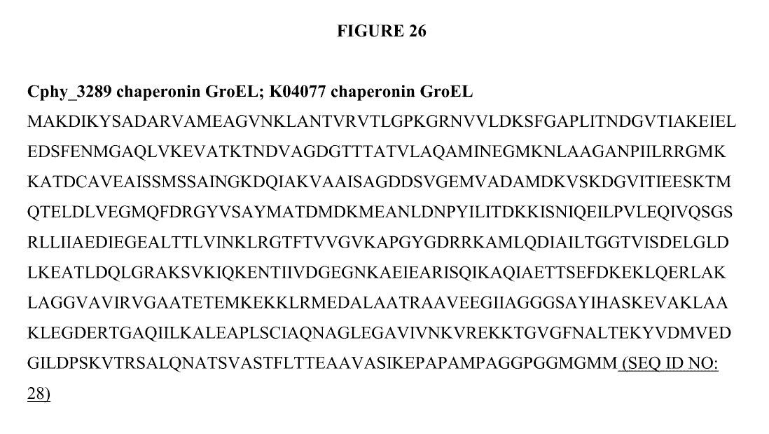

FIG. 27 illustrates the amino acid sequence of Cphy_{—}3290 chaperonin GroES.

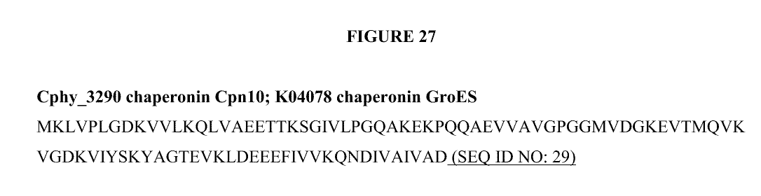

FIG. 28 illustrates the nucleic acid sequence of Cphy_{—}3510.

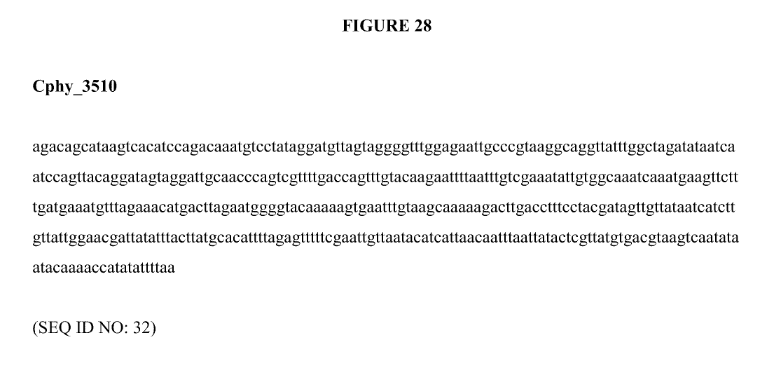

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The following description and examples illustrate embodiments of the present invention in detail. Generally, methods and compositions directed to saccharification and fermentation of various biomass substrates to one or more fermentive products are disclosed.

Unless characterized differently, technical and scientific terms used herein have the same meaning as commonly understood by one of ordinary skill in the art.

### DEFINITIONS

The term “enzyme reactive conditions” as used herein, refers to an environmental condition (i.e., such factors as temperature, pH, lack of inhibiting substances) which will permit the enzyme to function. Enzyme reactive conditions can be either in vitro, such as in a test tube, or in vivo, such as within a cell.

The term “about” as used herein, refers to a range that is 15% plus or minus from a stated numerical value within the context of the particular usage. For example, about 10 would include a range from 8.5 to 11.5.

The terms “function” and “functional” as used herein refer to biological or enzymatic function.

The term “gene” as used herein, refers to a unit of inheritance that occupies a specific locus on a chromosome and consists of transcriptional and/or translational regulatory sequences and/or a coding region and/or non-translated sequences (i.e., introns, 5′ and 3′ untranslated sequences). The term “host cell” includes an individual cell or cell culture which can be or has been a recipient of any recombinant vector(s) or isolated polynucleotide. Host cells include progeny of a single host cell, and the progeny can not necessarily be completely identical (in morphology or in total DNA complement) to the original parent cell due to natural, accidental, or deliberate mutation and/or change. A host cell includes cells transfected, transformed, or infected in vivo or in vitro with a recombinant vector or a polynucleotide. A host cell which comprises a recombinant vector is a recombinant host cell, recombinant cell, or recombinant microorganism.

The term “isolated” as used herein, refers to material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

The term “increased” or “increasing” as used herein, refers to the ability of one or more recombinant microorganisms to produce a greater amount of a given product or molecule (e.g., commodity chemical, biofuel, or intermediate product thereof) as compared to a control microorganism, such as an unmodified microorganism or a differently-modified microorganism. An “increased” amount is typically a “statistically significant” amount, and can include an increase that is 1, 2, 3, 4, 5, 6, 7, 8, 9, 10, 15, 20, 30 or more times (including all integers and decimal points in between, e.g., 1.5, 1.6, 1.7. 1.8, etc.) the amount produced by an unmodified microorganism or a differently modified microorganism.

The term “operably linked” as used herein means placing a gene under the regulatory control of a promoter, which then controls the transcription and optionally the translation of the gene. In one example for the construction of promoter/structural gene combinations, the genetic sequence or promoter is positioned at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, a regulatory sequence element can be positioned with respect to a gene to be placed under its control in the same position as the element is situated in its in its natural setting with respect to the native gene it controls.

The term “Constitutive promoter” refers to a polynucleotide sequence that induces transcription or is typically active, (i.e., promotes transcription), under most conditions, such as those that occur in a host cell. A constitutive promoter is generally active in a host cell through a variety of different environmental conditions.

The term “Inducible promoter” refers to a polynucleotide sequence that induces transcription or is typically active only under certain conditions, such as in the presence of a specific transcription factor or transcription factor complex, a given molecule factor (e.g., IPTG) or a given environmental condition (e.g., CO2concentration, nutrient levels, light, heat). In the absence of that condition, inducible promoters typically do not allow significant or measurable levels of transcriptional activity.

The terms “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

As will be understood by those skilled in the art, a polynucleotide sequence can include genomic sequences, extra-genomic and plasmid-encoded sequences and smaller engineered gene segments that express, or can be adapted to express, proteins, polypeptides, peptides and the like. Such segments can be naturally isolated, or modified synthetically by the hand of man.

Polynucleotides can be single-stranded (coding or antisense) or double-stranded, and can be DNA (genomic, cDNA or synthetic) or RNA molecules. In one embodiment additional coding or non-coding sequences can, be present within a polynucleotide. In another embodiment a polynucleotide can be linked to other molecules and/or support materials.

Polynucleotides can comprise a native sequence (i.e., an endogenous sequence) or can comprise a variant, or a biological functional equivalent of such a sequence. Polynucleotide variants can contain one or more base substitutions, additions, deletions and/or insertions, as further described below. In one embodiment a polynucleotide variant encodes a polypeptide with the same sequence as the native protein. In another embodiment a polynucleotide variant encodes a polypeptide with substantially similar enzymatic activity as the native protein. In another embodiment a polynucleotide variant encodes a protein with increased enzymatic activity relative to the native polypeptide. The effect on the enzymatic activity of the encoded polypeptide can generally be assessed as described herein.

A polynucleotide encoding a polypeptide can be combined with other DNA sequences, such as promoters, polyadenylation signals, additional restriction enzyme sites, multiple cloning sites, other coding segments, and the like, such that their overall length can vary considerably. In one embodiment the maximum length of a polynucleotide sequence which can be used to transform a microorganism is governed only by the nature of the recombinant protocol employed.

The terms “polynucleotide variant” and “variant” and the like refer to polynucleotides that display substantial sequence identity with any of the reference polynucleotide sequences or genes described herein, and to polynucleotides that hybridize with any polynucleotide reference sequence described herein, or any polynucleotide coding sequence of any gene or protein referred to herein, under low stringency, medium stringency, high stringency, or very high stringency conditions that are defined hereinafter and known in the art. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between) sequence identity with a reference polynucleotide described herein.

The terms “polynucleotide variant” and “variant” also include naturally-occurring allelic variants that encode these enzymes. Examples of naturally-occurring variants include allelic variants (same locus), homologs (different locus), and orthologs (different microorganism). Naturally occurring variants such as these can be identified and isolated using well-known molecular biology techniques including, for example, various polymerase chain reaction (PCR) and hybridization-based techniques as known in the art. Naturally-occurring variants can be isolated from any microorganism that encodes one or more genes having a suitable enzymatic activity described herein (e.g., C—C ligase, diol dehyodrogenase, pectate lyase, alginate lyase, diol dehydratase, transporter, etc.).

Non-naturally occurring variants can be made by mutagenesis techniques, including those applied to polynucleotides, cells, or microorganisms. The variants can contain nucleotide substitutions, deletions, inversions and insertions. Variation can occur in either or both the coding and non-coding regions. In certain aspects, non-naturally occurring variants can have been optimized for use in a given microorganism (e.g., E. coli), such as by engineering and screening the enzymes for increased activity, stability, or any other desirable feature. The variations can produce both conservative and non-conservative amino acid substitutions (as compared to the originally encoded product). For polynucleotide sequences, conservative variants include those sequences that, because of the degeneracy of the genetic code, encode the amino acid sequence of a reference polypeptide. Variant polynucleotide sequences also include synthetically derived nucleotide sequences, such as those generated, for example, by using site-directed mutagenesis but which still encode a biologically active polypeptide. Generally, variants of a reference polynucleotide sequence will have at least about 30%, 40% 50%, 55%, 60%, 65%, 70%, generally at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity with the reference polynucleotide sequence as determined by sequence alignment programs described elsewhere herein using default parameters. In one embodiment a variant polynucleotide sequence encodes a protein with substantially similar activity compared to a protein encoded by the respective reference polynucleotide sequence. Substantially similar activity means variant protein activity that is within +/−15% of the activity of a protein encoded by the respective reference polynucleotide sequence. In another embodiment a variant polynucleotide sequence encodes a protein with greater activity compared to a protein encoded by the respective reference polynucleotide sequence.

The terms “hybridizes under low stringency, hybridizes medium stringency, hybridizes high stringency, or hybridizes very high stringency conditions” as used herein, refers to conditions for hybridization and washing. Guidance for performing hybridization reactions can be found in Ausubel et al., “Current Protocols in Molecular Biology”, John Wiley & Sons Inc, 1994-1998, Sections 6.3.1-6.3.6. Aqueous and non-aqueous methods are described in that reference and either can be used.

The term “low stringency” as used herein, refers to conditions that include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also can include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

The term “Medium stringency” as used herein, refers to conditions that include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also can include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

The term “High stringency” as used herein, refers to conditions that include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also can include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

Due to the degeneracy of the genetic code, amino acids can be substituted for other amino acids in a protein sequence without appreciable loss of the desired activity (see Table 1 below). It is thus contemplated that various changes can be made in the peptide sequences of the disclosed protein sequences, or their corresponding nucleic acid sequences without appreciable loss of the biological activity.

In making such changes, the hydropathic index of amino acids can be considered. The importance of the hydropathic amino acid index in conferring interactive biological function on a protein is generally understood in the art (Kyte and Doolittle, J. Mol. Biol., 157: 105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Amino acids have been assigned a hydropathic index on the basis of their hydrophobicity and charge characteristics. These are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

It is known in the art that certain amino acids can be substituted by other amino acids having a similar hydropathic index or score and result in a protein with similar biological activity, i.e., still obtain a biologically-functional protein. In one embodiment, the substitution of amino acids whose hydropathic indices are within +/−0.2 is preferred, those within +/−0.1 are more preferred, and those within +/−0.5 are most preferred.

It is also understood in the art that the substitution of like amino acids can be made effectively on the basis of hydrophilicity. U.S. Pat. No. 4,554,101 (Hopp, which is herein incorporated by reference in its entirety) states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0.+−0.1); serine (+0.3); asparagine/glutamine (+0.2); glycine (0); threonine (−0.4); proline (−0.5.+−0.1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

It is understood that an amino acid can be substituted by another amino acid having a similar hydrophilicity score and still result in a protein with similar biological activity, i.e., still obtain a biologically functional protein. In one embodiment the substitution of amino acids whose hydropathic indices are within +/−0.2 is preferred, those within +/−0.1 are more preferred, and those within. +/−0.5 are most preferred.

As outlined above, amino acid substitutions can be based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine, and isoleucine. Changes which are not expected to be advantageous can also be used if these resulting proteins have the same or improved characteristics, relative to the unmodified polypeptide from which they are engineered.

In one embodiment a polynucleotide comprises codons in its protein coding sequence that are optimized to increase the thermostability of an mRNA transcribed from the polynucleotide. In one embodiment this optimization does not change the amino acid sequence encoded by the polynucleotide. In another embodiment a polynucleotide comprises codons in its protein coding sequence that are optimized to increase translation efficiency of an mRNA from the polynucleotide in a host cell. In one embodiment this optimization does not change the amino acid sequence encoded by the polynucleotide.

The RNA codon table below (Table 1) shows the 64 codons and the amino acid for each. The direction of the mRNA is 5′ to 3′.

TABLE 1

| 2nd base | | | |
|---|---|---|---|
| U | C | A | G |

| | | | | | |
|---|---|---|---|---|---|
| 1st | U | UUU (Phe/F) | UCU (Ser/S) | UAU (Tyr/Y) | UGU (Cys/C) |
| base | | Phenylalanine | Serine | Tyrosine | Cysteine |
| | | UUC (Phe/F) | UCC (Ser/S) | UAC (Tyr/Y) | UGC (Cys/C) |
| | | Phenylalanine | Serine | Tyrosine | Cysteine |
| | | UUA (Leu/L) | UCA (Ser/S) | UAA Ochre | UGA Opal |
| | | Leucine | Serine | (Stop) | (Stop) |
| | | UUG (Leu/L) | UCG (Ser/S) | UAG Amber | UGG (Trp/W) |
| | | Leucine | Serine | (Stop) | Tryptophan |
| | C | CUU (Leu/L) | CCU (Pro/P) | CAU (His/H) | CGU (Arg/R) |
| | | Leucine | Proline | Histidine | Arginine |
| | | CUC (Leu/L) | CCC (Pro/P) | CAC (His/H) CGC (Arg/R) | |
| | | Leucine | Proline | Histidine | Arginine |
| | | CUA (Leu/L) | CCA (Pro/P) | CAA (Gln/Q) | CGA (Arg/R) |
| | | Leucine | Proline | Glutamine | Arginine |
| | | CUG (Leu/L) | CCG (Pro/P) | CAG (Gln/Q) CGG (Arg/R) | |
| | | Leucine | Proline | Glutamine | Arginine |
| | A | AUU (Ile/I) | ACU (Thr/T) | AAU (Asn/N) | AGU (Ser/S) |
| | | Isoleucine | Threonine | Asparagine | Serine |
| | | AUC (Ile/I) | ACC (Thr/T) | AAC (Asn/N) | AGC (Ser/S) |
| | | Isoleucine | Threonine | Asparagine | Serine |
| | | AUA (Ile/I) | ACA (Thr/T) | AAA (Lys/K) | AGA (Arg/R) |
| | | Isoleucine | Threonine | Lysine | Arginine |
| | | AUG^{[A]} (Met/M) | ACG (Thr/T) | AAG (Lys/K) | AGG (Arg/R) |
| | | Methionine | Threonine | Lysine | Arginine |
| | G | GUU (Val/V) | GCU (Ala/A) | GAU (Asp/D) | GGU (Gly/G) |
| | | Valine | Alanine | Aspartic acid | Glycine |
| | | GUC (Val/V) | GCC (Ala/A) | GAC (Asp/D) | GGC (Gly/G) |
| | | Valine | Alanine | Aspartic acid | Glycine |
| | | GUA (Val/V) | GCA (Ala/A) | GAA (Glu/L) | GGA (Gly/G) |
| | | Valine | Alanine | Glutamic acid | Glycine |
| | | GUG (Val/V) | GCG (Ala/A) | GAG (Glu/L) | GGG (Gly/G) |
| | | Valine | Alanine | Glutamic acid | Glycine |

^{A}The codon AUG both codes for methionine and serves as an initiation site: the first AUG in an mRNA's coding region is where translation into protein begins.

In one embodiment a method disclosed which uses variants of full-length polypeptides having any of the enzymatic activities described herein, truncated fragments of these full-length polypeptides, variants of truncated fragments, as well as their related biologically active fragments. Typically, biologically active fragments of a polypeptide can participate in an interaction, for example, an intra-molecular or an inter-molecular interaction. An inter-molecular interaction can be a specific binding interaction or an enzymatic interaction (e.g., the interaction can be transient and a covalent bond is formed or broken). Biologically active fragments of a polypeptide/enzyme an enzymatic activity described herein include peptides comprising amino acid sequences sufficiently similar to, or derived from, the amino acid sequences of a (putative) full-length reference polypeptide sequence. Typically, biologically active fragments comprise a domain or motif with at least one enzymatic activity, and can include one or more (and in some cases all) of the various active domains. A biologically active fragment of a an enzyme can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, or 50% of an activity of the wild-type polypeptide from which it is derived.

The term “exogenous” as used herein, refers to a polynucleotide sequence or polypeptide that does not naturally occur in a given wild-type cell or microorganism, but is typically introduced into the cell by a molecular biological technique, i.e., engineering to produce a recombinant microorganism. Examples of “exogenous” polynucleotides include vectors, plasmids, and/or man-made nucleic acid constructs encoding a desired protein or enzyme.

The term “endogenous” as used herein, refers to naturally-occurring polynucleotide sequences or polypeptides that can be found in a given wild-type cell or microorganism. For example, certain naturally-occurring bacterial or yeast species do not typically contain a benzaldehyde lyase gene, and, therefore, do not comprise an “endogenous” polynucleotide sequence that encodes a benzaldehyde lyase. In this regard, it is also noted that even though a microorganism can comprise an endogenous copy of a given polynucleotide sequence or gene, the introduction of a plasmid or vector encoding that sequence, such as to over-express or otherwise regulate the expression of the encoded protein, represents an “exogenous” copy of that gene or polynucleotide sequence. Any of the of pathways, genes, or enzymes described herein can utilize or rely on an “endogenous” sequence, or can be provided as one or more “exogenous” polynucleotide sequences, and/or can be used according to the endogenous sequences already contained within a given microorganism.

The term “sequence identity” for example, comprising a “sequence 50% identical to,” as used herein, refers to the extent that sequences are identical on a nucleotide-by-nucleotide basis or an amino acid-by-amino acid basis over a window of comparison. Thus, a “percentage of sequence identity” can be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides can each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window can comprise additions or deletions (i.e., gaps) of about 20% or less as compared to the reference sequence (which does not comprise additions or deletions) for optimal alignment of the two sequences. Optimal alignment of sequences for aligning a comparison window can be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also can be made to the BLAST family of programs as for example disclosed by Altschul et al., 1997, Nucl. Acids Res. 25:3389, which is herein incorporated by reference in its entirety. A detailed discussion of sequence analysis can be found in Unit 19.3 of Ausubel et al., “Current Protocols in Molecular Biology”, John Wiley & Sons Inc, 1994-1998, Chapter 15, which is herein incorporated by reference in its entirety.

The term “transformation” as used herein, refers to the permanent, heritable alteration in a cell resulting from the uptake and incorporation of foreign DNA into the host-cell genome. This includes the transfer of an exogenous gene from one microorganism into the genome of another microorganism as well as the addition of additional copies of an endogenous gene into a microorganism.

The term “vector” as used herein, refers to a polynucleotide molecule, such as a DNA molecule. It can be derived, from a plasmid, bacteriophage, yeast or virus, into which a polynucleotide can be inserted or cloned. A vector can contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector can comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. A vector can be one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The terms “wild-type” and “naturally-occurring” as used herein are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene.

The term “fuel” or “biofuel” as used herein has its ordinary meaning as known to those skilled in the art and can include one or more compounds suitable as liquid fuels, gaseous fuels, reagents, chemical feedstocks and includes, but is not limited to, hydrocarbons, hydrogen, methane, hydroxy compounds such as alcohols (e.g. ethanol, butanol, propanol, methanol, etc.), and carbonyl compounds such as aldehydes and ketones (e.g. acetone, formaldehyde, 1-propanal, etc.).

The terms “fermentation end-product” or “end-product” as used herein has its ordinary meaning as known to those skilled in the art and can include one or more biofuels, chemical additives, processing aids, food additives, organic acids (e.g. acetic, lactic, formic, citric acid etc.), derivatives of organic acids such as esters (e.g. wax esters, glycerides, etc.) or other functional compounds. These end-products include, but are not limited to, an alcohol, ethanol, butanol, methanol, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases and can be present as a pure compound, a mixture, or an impure or diluted form.

Various end-products can be produced through saccharification and fermentation using enzyme-enhancing products and processes. Examples of end-products include but are not limited to methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl) butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, ddodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid or formic acid.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The cellular activity, including cell growth can be growing aerobic, microaerophilic, or anaerobic. The cells can be in any phase of growth, including lag (or conduction), exponential, transition, stationary, death, dormant, vegetative, sporulating, etc.

The term “external source” as it relates to a quantity of an enzyme or enzymes provided to a product or a process means that the quantity of the enzyme or enzymes is not produced by a microorganism in the product or process. An external source of an enzyme can include, but is not limited to an enzyme provided in purified form, cell extracts, culture medium or an enzyme obtained from a commercially available source.

The term “plant polysaccharide” as used herein has its ordinary meaning as known to those skilled in the art and can comprise one or more carbohydrate polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or non-regular pattern. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be used as a carbon source by the microorganism, including monomers, dimers, and polymers of these compounds including two or more of these compounds. In some cases, the microorganism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be used by the microorganism at hand. For some microorganisms, this would include conversion to monosaccharides, disaccharides, trisaccharides, and oligosaccharides of up to about seven monomer units, as well as similar sized chains of sugar derivatives and combinations of sugars and sugar derivatives. For some microorganisms, the allowable chain-length can be longer (e.g. 8, 9, 10, 11, 12, 13, 14, 15, 16, 17, 18, 19, or 20 monomer units or more) and for some microorganisms the allowable chain-length can be shorter (e.g. 1, 2, 3, 4, 5, 6, or 7 monomer units).

The term “carbonaceous biomass” as used herein has its ordinary meaning as known to those skilled in the art and can include one or more biological material that can be converted into a biofuel, chemical or other product. Carbonaceous biomass can comprise municipal waste, wood, plant material, plant extract, a natural or synthetic polymer, or a combination thereof.

Plant matter can include, but is not limited to, woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, bamboo, algae and material derived from these.

“Biomass” can include, but is not limited to, woody or non-woody plant matter, aquatic or marine biomass, fruit-based biomass such as fruit waste, and vegetable-based biomass such as vegetable waste, and animal based biomass among others. Examples of aquatic or marine biomass include, but are not limited to, kelp, other seaweed, algae, and marine microflora, microalgae, sea grass, salt marsh grasses such as Spartina sp. or Phragmites sp. and the like. In one embodiment, biomass does not include fossilized sources of carbon, such as hydrocarbons that are typically found within the top layer of the Earth's crust (e.g., natural gas, nonvolatile materials composed of almost pure carbon, like anthracite coal, etc.).

Examples of fruit and/or vegetable biomass include, but are not limited to, any source of pectin such as plant peel and pomace including citrus, orange, grapefruit, potato, tomato, grape, mango, gooseberry, carrot, sugar-beet, and apple, among others. In one embodiment plant matter is characterized by the chemical species present, such as proteins, polysaccharides and oils. In one embodiment plant matter includes agricultural waste byproducts or side streams such as pomace, corn steep liquor, corn steep solids, distillers grains, peels, pits, fermentation waste, straw, lumber, sewage, garbage and food leftovers. These materials can come from farms, forestry, industrial sources, households, etc. In another embodiment biomass comprises animal matter, including, for example milk, meat, fat, animal processing waste, and animal waste. The term “feedstock” is frequently used to refer to biomass being used for a process, such as those described herein.

Examples of polysaccharides, oligosaccharides, monosaccharides or other sugar components of biomass include, but are not limited to, alginate, agar, carrageenan, fucoidan, floridean starch, pectin, gluronate, mannuronate, mannitol, lyxose, cellulose, hemicellulose, glycerol, xylitol, glucose, mannose, galactose, xylose, xylan, mannan, arabinan, arabinose, glucuronate, galacturonate (including di- and tri-galacturonates), rhamnose, and the like.

The term “broth” as used herein has its ordinary meaning as known to those skilled in the art and can include the entire contents of the combination of soluble and insoluble matter, suspended matter, cells and medium, such as for example the entire contents of a fermentation reaction can be referred to as a fermentation broth.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity (e.g. g/L/d) is different from “titer” (e.g. g/L) in that productivity includes a time term, and titer is analogous to concentration.

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency or yield is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art. For substrates comprising a mixture of different carbon sources such as found in biomass (xylan, xylose, glucose, cellobiose, arabinose cellulose, hemicellulose etc.), the theoretical maximum conversion efficiency of the biomass to ethanol is an average of the maximum conversion efficiencies of the individual carbon source constituents weighted by the relative concentration of each carbon source. In some cases, the theoretical maximum conversion efficiency is calculated based on an assumed saccharification yield. In one embodiment, given carbon source comprising 10 g of cellulose, the theoretical maximum conversion efficiency can be calculated by assuming saccharification of the cellulose to the assimilable carbon source glucose of about 75% by weight. In this embodiment, 10 g of cellulose can provide 7.5 g of glucose which can provide a maximum theoretical conversion efficiency of about 7.501% or 3.8 g of ethanol. In other cases, the efficiency of the saccharification step can be calculated or determined, i.e., saccharification yield. Saccharification yields can include between about 10-100%, about 20-90%, about 30-80%, about 40-70% or about 50-60%, such as about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% for any carbohydrate carbon sources larger than a single monosaccharide subunit.

The saccharification yield takes into account the amount of ethanol, and acidic products produced plus the amount of residual monomeric sugars detected in the media. The ethanol figures resulting from media components are not adjusted in this experiment. These can account for up to 3 g/l ethanol production or equivalent of up to 6 g/l sugar as much as +/−10%-15% saccharification yield (or saccharification efficiency). For this reason the saccharification yield % can be greater than 100% for some plots.

The terms “Pretreatment” or “pretreated” as used herein refer to any mechanical, chemical, thermal, biochemical process or combination of these processes whether in a combined step or performed sequentially, that achieves disruption or expansion of a biomass so as to render the biomass more susceptible to attack by enzymes and/or microbes. In some embodiments, pretreatment can include removal or disruption of lignin so is to make the cellulose and hemicellulose polymers in the plant biomass more available to cellulolytic enzymes and/or microbes, for example, by treatment with acid or base. In some embodiments, pretreatment can include the use of a microorganism of one type to render plant polysaccharides more accessible to microorganisms of another type. In some embodiments, pretreatment can also include disruption or expansion of cellulosic and/or hemicellulosic material. Steam explosion, and ammonia fiber expansion (or explosion) (AFEX) are well known thermal/chemical techniques. Hydrolysis, including methods that utilize acids and/or enzymes can be used. Other thermal, chemical, biochemical, enzymatic techniques can also be used.

The terms “fed-batch” or “fed-batch fermentation” as used herein has its ordinary meaning as known to those skilled in the art and can include a method of culturing microorganisms where nutrients, other medium components, or biocatalysts (including, for example, enzymes, fresh microorganisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

The term “SSF” as used herein, refers to simultaneous saccharification fermentation. The term “SHF” means sequential hydrolysis followed by subsequent fermentation.

A term “phytate” as used herein has its ordinary meaning as known to those skilled in the art can be include phytic acid, its salts, and its combined forms as well as combinations of these.

The term “recombinant” as used herein, refers to a microorganism is genetically modified to comprise one or more heterologous or endogenous nucleic acid molecules. Such nucleic acid molecules can be comprised extrachromosomally or integrated into the chromosome of a microorganism. The term “non-recombinant” means a microorganism is not genetically modified. For example, a recombinant microorganism can be modified to overexpress an endogenous gene encoding an enzyme through modification of promoter elements (e.g., replacing an endogenous promoter element with a constitutive or highly active promoter). Alternatively, a recombinant microorganism can be modified by introducing a heterologous or another copy of an endogenous nucleic acid molecule encoding a protein that is not otherwise expressed in the host microorganism.

The term “sugar compounds” as used herein has its ordinary meaning as known to those skilled in the art and can include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

Generally, compositions and methods are provided for enzyme conditioning of feedstock or biomass to allow saccharification and fermentation to one or more industrially useful fermentive end-products.

In one aspect one or more products are provided for production of a biofuel from biomass.

Enzyme-assisted fermentation Involves mixtures of enzymes, derived from several microorganisms, which are added during saccharification steps in order to improve end-product yield during fermentation by increasing hydrolysis. This can be an expensive proposition since mixes of enzymes are expensive and affect the final cost of a biofuel produced by a fermentation processes.

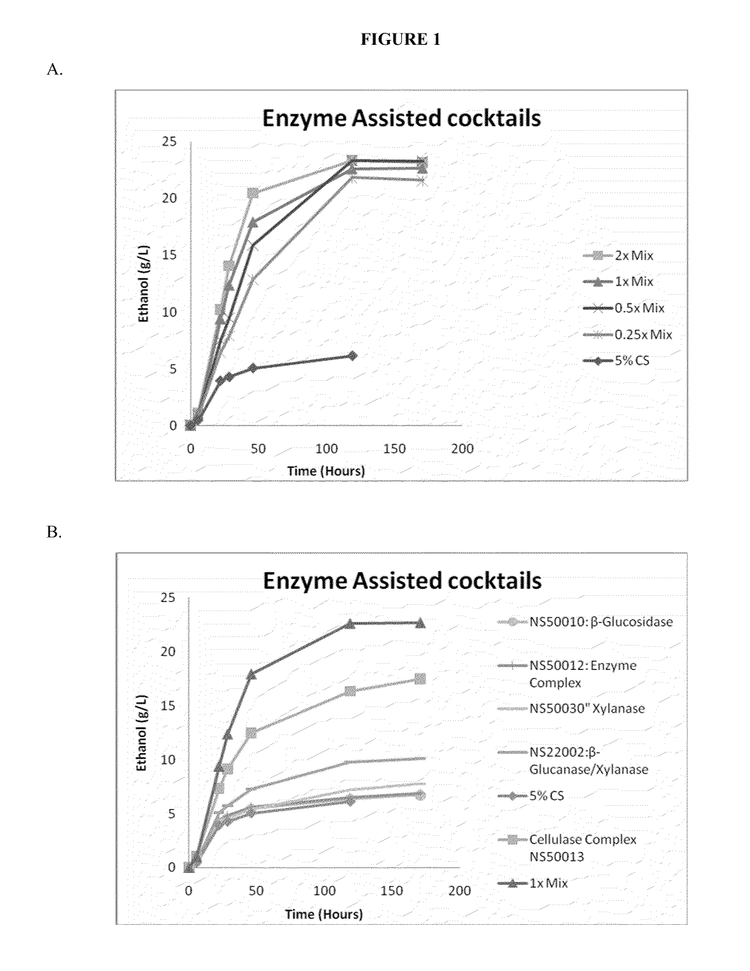

As demonstrated in the disclosure herein, it was observed that for certain enzyme-assisted cocktails tested at various concentrations, certain enzymes did not substantially affect saccharification and fermentation of the biomass tested. Furthermore, the enzyme cocktail (Novozymes A/S, Krogshoejvej, 36, 2880, Bagsvaerd, Denmark) is recommended for use at 1×. However, by increasing dosing of enzymes to double that of recommended dosage (FIG. 1A demonstrates that various concentrations of a cocktail mix, i.e., 0.25× to 2×) no significant difference in ethanol production is observed in a simultaneous saccharification and fermentation. At 25% the recommended dosage more than 80% fermentation (as compared to full dosing) is observed. In addition, the non-enzyme assisted fermentation achieved only about 20% of theoretical yield.

FIG. 1B illustrates the performance of the individual enzyme components of the cocktail mix used in FIG. 1A, and were supplied at 1×. The results demonstrate that none of the individual components performed as well as the cocktail mixture. However, the results also demonstrate that individually, β-glucosidase, xylanase and hemicellulase did not enhance ethanol production. Cellulase alone (NS50013, Novozymes, supra) resulted in greater than 63% yield relative to theoretical yield. Furthermore, a β-glucanase/xylanase mix that contains cellulase and hemicellulase activity also enhanced ethanol production.

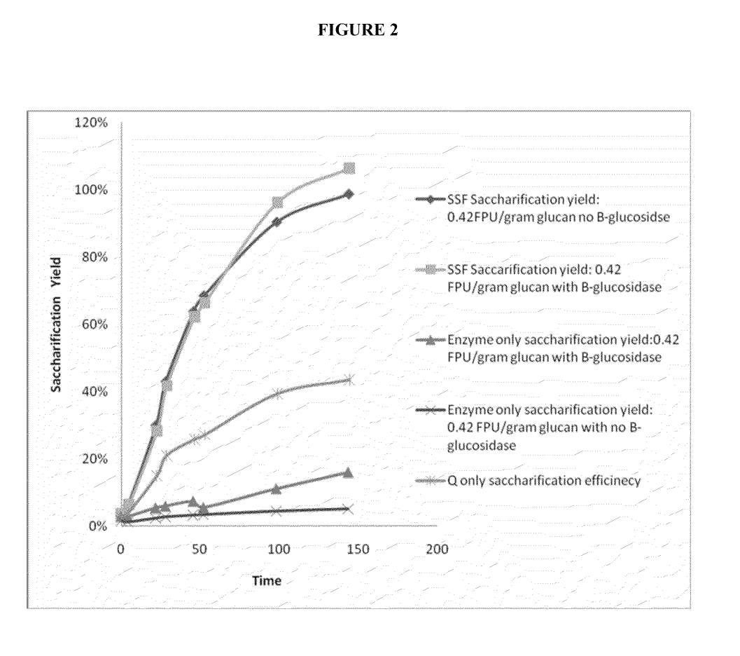

A synergistic effect is observed with respect to saccharification yield when utilizing an organism that is capable of direct saccharification, e.g., C. phytofermentans, and an external source of a cellulase to achieve complete saccharification and fermentation (FIG. 2). In other words, the presence of cellulase enhances the effects of C. phytofermentans saccharification so that the resulting hydrolysis is better than expected from the addition of the two.

Saccharification yield of feedstock contacted with C. phytofermentans is generally not affected by the addition of an external source of β-glucosidase. The non-microbe innoculated reactions exhibited poor saccharification yield. The pH of the reactions was at about pH 6.5 and the temperature was about 35° C. Saccharification yield can be enhanced by decreasing pH to about 5.4 and increasing temperature to about 65° C. This result indicates that β-glucosidase can be excluded when adding an external source of enzymes to enhance saccharification and fermentation of feedstock.

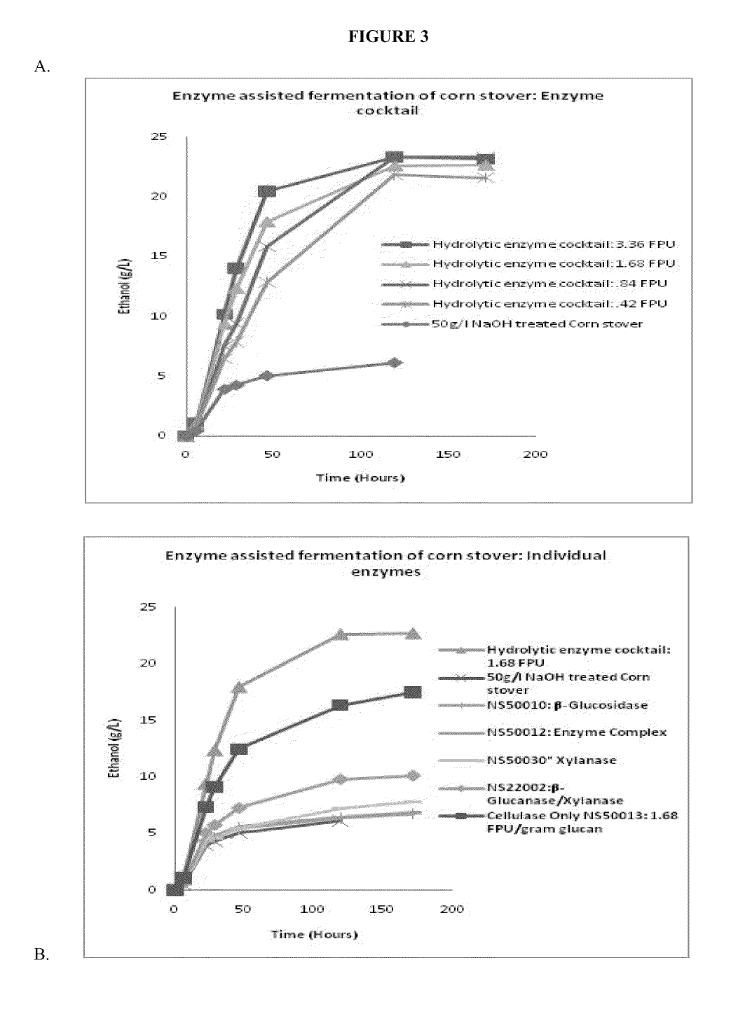

Furthermore, various dosages of a hydrolytic enzyme cocktail (from 0.25 to 2×) result in theoretical ethanol conversions (FIG. 3A) that are similar. In addition, the curves for individual enzyme augmentations illustrate the impact of cellulase additions and also clearly demonstrate the ineffectiveness of B-glucosidase, xylanase and hemicelluloses additions (FIG. 3B). FIGS. 3A and 3B correspond to the experiments illustrated in FIGS. 1A and 1B, but are different in providing FPUs (Filter Paper Units) for hydrolytic enzymes or cellulases that are present in the cocktail mix.

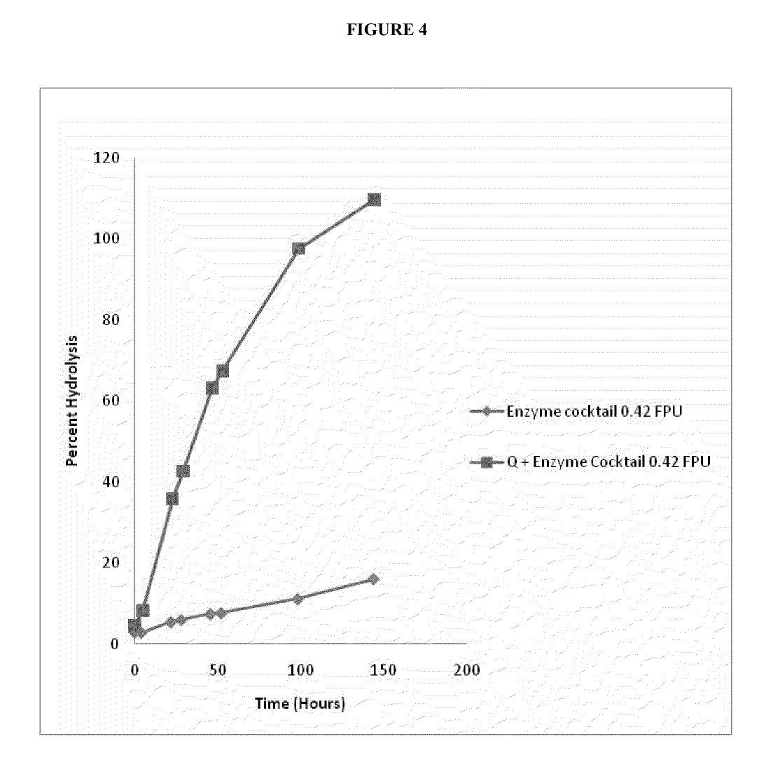

When examining saccharification with and without a microbe, the extent of hydrolysis using enzyme alone resulted in a reduced saccharification yield, i.e., 16.8% (FIG. 4).

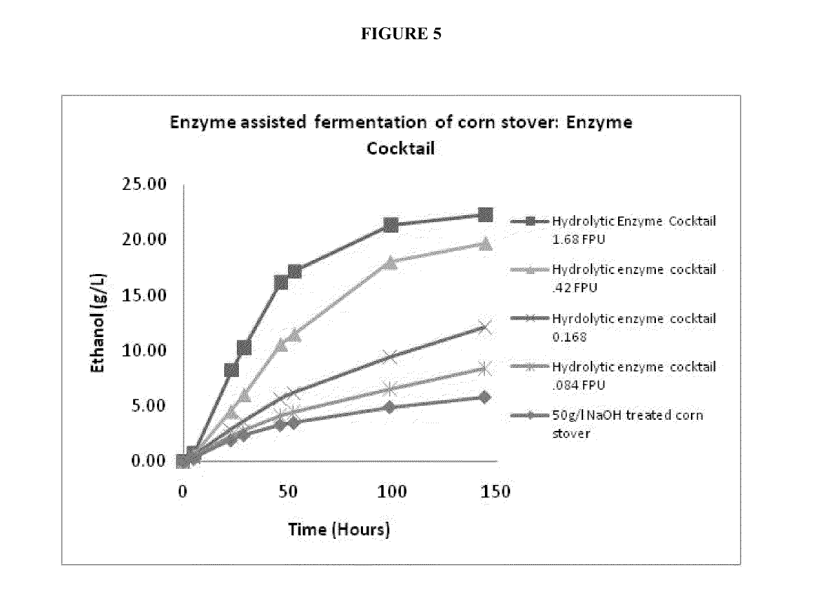

Furthermore, reducing dosages from 1.68 FPU/gram of glucan to lower than about 0.4 FPU resulted in proportional reduction to 0.168 FPU and 0.084 FPU loadings (FIG. 5).

Utilization of cellulases as the external source for enhancing saccharification and the fermentation yield, provides a substantial improvement in rate and yield of cellulose utilization by supplementation of additional endo-glucanase activity. The addition of a cellulase enzyme alone obviates the requirement of other enzymes for the saccharification of polysaccharides in a C5/C6 fermenting microorganism. In fact, small amounts of a cellulase synergistically enhance the rate of hydrolysis of C6 sugars so that biofuel production is more rapid and more efficient. This discovery will significantly reduce the cost of producing biofuels such as ethanol, hydrogen, methane and the like.

Clostridium phytofermentans is one microorganism that can simultaneously hydrolyze and ferment hexose (C6) and pentose (C5) polysaccharides. This microorganism has a complement of enzymes to adapt to any biomass substrate. However, the hydrolysis of cellulose in the naturally-occurring microorganism is initially slower than desirable for cost-effective production of biofuels. Unlike other microorganisms, β-glucosidase does not enhance the hydrolysis of cellulose in this microorganism. In one embodiment there is substantial improvement in the rate and yield of cellulose utilization for a microrganism by upregulation or supplementation of additional endo-glucanase activity. The addition of small amounts of a cellulase enzyme alone synergistically enhance the rate of hydrolysis of C6 sugars in C. phytofermentans and increases the yield of fermentation end-products.

In one embodiment, a product for production of a biofuel comprises: a carbonaceous biomass, a microorganism that is capable of direct hydrolysis and fermentation of said biomass, and an external source of one or more enzymes that are capable of enhancing said hydrolysis, wherein said one or more enzymes do not include a xylanase, a hemicellulase, a glucanase or glucosidase, and wherein said external source is not said microorganism.

In another embodiment a product for production of a biofuel is provided comprising: a carbonaceous biomass, a microorganism that is capable of direct hydrolysis and fermentation of said biomass, wherein said microorganism is modified to provide enhanced activity of one or more cellulases.

In another embodiment a product for production of fermentive end-products comprises: (a) a fermentation vessel comprising a carbonaceous biomass; (b) a microorganism that is capable of direct hydrolysis and fermentation of said biomass; and (c) a source of one or more enzymes that is external to said microorganism, wherein said one or more enzymes do not include a xylanase, a hemicellulase, a glucanase or glucosidase; wherein the fermentation vessel is adapted to provide suitable conditions for fermentation of one or more carbohydrates into fermentive end-products.

In one embodiment a microorganism is capable of direct fermentation of C5 (five carbon chain polysaccharide) and/or C6 (six carbon chain polysaccharide) carbohydrates. In one embodiment, such a capability is achieved through modifying the microorganism to express one or more genes encoding proteins associated with C5 and C6 carbohydrate metabolization.

Microorganisms that can be used in a composition or method disclosed herein include but are not limited to bacteria, yeast or fungi. In some embodiments, two or more different microorganisms can be used during saccharification and/or fermentation processes to produce an end-product. Microorganisms used can be recombinant, non-recombinant or wild type.

In one embodiment, a microorganism used in a composition or method disclosed herein is a strain of Clostridia. The strain can be C. acetobutylicum, C. bejeirinckii, C. saccharoperbutylacetonicum, C. butylicum, C. butylicum, C. perfringens, C. tetani, C. sporogenes, C. thermocellum, C. saccarolyticum (now Thermoanaerobacter saccarolyticum), C. thermosulfurogenes (now Thermoanaerobacter thermosulfurigenes), C. thermohydrosulfuricum (now Thermoanaerobacter ethanolicus), and C. phytofermentans. In one embodiment, the microorganism is Clostridium phytofermentans.

In one embodiment a microorganism can be modified to comprise one or more heterologous polynucleotides that enhance enzyme function. In one embodiment, enzymatic function is increased for one or more cellulase enzymes or other hydrolases.

In another embodiment a microrganism can be modified to comprise one or more additional copies of an endogenous polynucleotide that encodes a protein. In one embodiment the protein is a cellulase enzyme. In another embodiment the protein is a hydrolase enzyme. In another embodiment a microrganism can be modified to comprises more than one additional copies of an endogenous polynucleotide that encodes a protein.

In another embodiment a microorganism can be capable of uptake of one or more complex carbohydrates from a biomass (e.g., biomass comprises a higher concentration of oligomeric carbohydrates relative to monomeric carbohydrates).

In one embodiment, one or more enzymes from an external source (e.g., enzymes provided in purified form, cell extracts, culture medium or commercially available source) is added to a product or process disclosed herein.

In one embodiment a product or a process is disclosed for producing an end-product from biomass, a carbonaceous biomass is contacted with: (1) a microorganism that is capable of direct hydrolysis and fermentation of said biomass, and/or (2) an external source of one or more enzymes that are capable of enhancing said hydrolysis, wherein said one or more enzymes do not include a xylanase, a hemicellulase, a glucanase or glucosidase, and wherein said external source is not said microorganism; thereby producing a fermentive medium; and allowing sufficient time for said hydrolysis and fermentation to produce a biofuel.

Furthermore, a microorganism that is used with or without an external source of one or more enzymes, can itself be modified to enhance enzyme function of one or more enzymes associated with hydrolyzation of biomass, fermentation of a polysaccharide or monosaccharide, or both.

Enzyme activity can also be enhanced by modifying conditions in a reaction vessel, including but not limited to time, pH of a culture medium, temperature, concentration of nutrients and/or catalyst, or a combination thereof.

A reaction vessel can be configured to separate one or more desired end-products.

Enzymes added externally can be in an amount from about 0.5 FPU/gram cellulose to about 20 FPU/gram cellulose, about 0.5 FPU/gram cellulose to about 40 FPU/gram cellulose, about 10 to about 30 FPU/gram cellulose, about 15 to about 25 FPU/gram cellulose, or about 20 to about 40 FPU/gram cellulose. In various embodiments, one or more cellulase enzymes can be added to a product or process disclosed herein to enhance saccharification and increase substrates available for fermentation.

In one embodiment a modified microorganism can have enhanced activity of an enzyme that is equivalent to addition of said cellulases in an amount sufficient to provide activity of about 0.5 FPU/gram cellulose to about 20 FPU/gram cellulose, about 0.5 FPU to about 40 FPU/gram cellulose, about 10 to about 30 FPU/gram cellulose, about 15 to about 25 FPU/gram cellulose, or about 20 to about 40 FPU/gram cellulose.

In one embodiment a product or process can provide hydrolysis of a biomass resulting in a greater concentration of cellobiose relative to monomeric carboyhdrates. Such monomeric carbohydrates can comprise xylose and arabinose.

In one embodiment, batch fermentation with a microorganism and of a mixture of hexose and pentose saccharides can provides uptake rates of about 0.1, 0.2, 0.4, 0.5, 0.6 0.7, 0.8, 1, 2, 3, 4, 5, or about 6 g/L/h or more of hexose (e.g. glucose, cellulose, cellobiose etc.), and about 0.1, 0.2, 0.4, 0.5, 0.6 0.7, 0.8, 1, 2, 3, 4, 5, or about 6 g/L/h or more of pentose (xylose, xylan, hemicellulose etc.). For example, C. phytofermentans is capable of direct fermentation of C5 and C6 sugars.

In another embodiment a product or process disclosed herein can produce about 15 g/L, about 20 g/L, about 25 g/L, about 30 g/L, about 35 g/L, about 40 g/L, about 45 g/L, about 50 g/L, about 60 g/L, about 70 g/L, about 80 g/L, or about 100 g/L production of ethanol. Such levels of ethanol can be observed in 10, 20, 30, 40, 50 or 60 hours of fermentation. In some embodiments the ethanol productivities provided by a process of the disclosed herein is due to the simultaneous fermentation of hexose and pentose saccharides.

Production of high levels of alcohol from biomass requires the ability for the microorganism to thrive generally in the presence of elevated alcohol levels, the ability to continue to produce alcohol without undue inhibition or suppression by the alcohol and/or other components present, and the ability to efficiently convert the multitude of different hexose and pentose carbon sources found in a biomass feedstock.

Fermentation at reduced pH and/or with the addition of fatty acids can result in about a three to five to 10 fold or higher increase in the ethanol production rate. In some embodiments, simultaneous fermentation of both hexose and pentose saccharides can also enable increases in ethanol productivity and/or yield. In some cases, the simultaneous fermentation of hexose and pentose carbohydrate substrates can be used in combination with fermentation at reduced pH and/or with the addition of fatty acids to further increase productivity, and/or yield.

Biomass

In some embodiments, a microorganism is contacted with pretreated or non-pretreated biomass containing cellulosic, hemicellulosic, and/or lignocellulosic material. Additional nutrients can be present or added to the biomass material to be processed by the microorganism including nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, and/or mineral supplements. In some embodiments, one or more additional lower molecular weight carbon sources can be added or be present such as glucose, sucrose, maltose, corn syrup, lactic acid, etc. Such lower molecular weight carbon sources can serve multiple functions including providing an initial carbon source at the start of the fermentation period, help build cell count, control the carbon/nitrogen ratio, remove excess nitrogen, or some other function.

In some embodiments aerobic/anaerobic cycling is employed for the bioconversion of cellulosic/lignocellulosic material to fuels and chemicals. In some embodiments, the anaerobic microorganism can ferment biomass directly without the need of a pretreatment. In certain embodiments, feedstocks are contacted with biocatalysts capable of breaking down plant-derived polymeric material into lower molecular weight products that can subsequently be transformed by biocatalysts to fuels and/or other desirable chemicals.

In some embodiments, a process for simultaneous saccharification and fermentation of cellulosic solids from biomass into biofuel or another end-product is provided. The process comprises treating the biomass in a closed container with a microorganism under conditions where the microorganism produces saccharolytic enzymes sufficient to substantially convert the biomass into monosaccharides and disaccharides. The organism subsequently converts the monosaccharides and disaccharides into ethanol and/or another biofuel or product.

In an alternative embodiment, a process for saccharification and fermentation comprises treating the biomass in a container with the microorganism and adding one or more enzymes before, concurrent or after contacting the biomass with the microorganism, wherein the enzymes added aid in the breakdown or detoxification of carbohydrates or lignocellulosic material.

In some instances, enzymes added do not include a xylanase, a hemicellulase, a glucanase or glucosidase. In other embodiments, the amount of exogenous cellulase is greatly reduced, one-quarter or less of the amount normally added to a fermentation wherein the organism cannot saccharify the biomass.

Examples of second cultures include but are not limited to Saccharomyces cerevisiae, Clostridia species such as C. thermocellum, C. acetobutylicum, and C. cellovorans, and Zymomonas mobilis.

In one embodiment, a process of producing a biofuel from a lignin-containing biomass is provided. In one embodiment the process comprises: 1) contacting the lignin-containing biomass with an aqueous alkaline solution at a concentration sufficient to hydrolyze at least a portion of the lignin-containing biomass; 2) neutralizing the treated biomass to a pH between 5 to 9 (e.g. 5.5, 6, 6.5, 7, 7.5, 8, 8.5, or 9); 3) treating the biomass in a closed container with a Clostridium phytofermentans bacterium under conditions wherein the Clostridium phytofermentans, optionally with the addition of one or more enzymes to the container, substantially converts the treated biomass into monosaccharides and disaccharides, and/or biofuel or other fermentation end-product; and 4) optionally, introducing a culture of a second microorganism wherein the second microorganism is capable of substantially converting the monosaccharides and disaccharides into biofuel.

Modification to Enhance Enzyme Activity

In one embodiment one or more modifications hydrolysis and/or fermentation conditions can be implemented to enhance end-product production. Examples of such modifications include genetic modification to enhance enzyme activity in a microorganism that already comprises genes for encoding one or more target enzymes, introducing one or more heterogeneous nucleic acid molecules into a host microorganism to express and enhance activity of an enzyme not otherwise expressed in the host, modifying physical and chemical conditions to enhance enzyme function (e.g., modifying and/or maintaining a certain temperature, pH, nutrient concentration, temporal), or a combination of one or more such modifications.

Genetic Modification

In one embodiment, a microorganism can be genetically modified to enhance enzyme activity of one or more enzymes, including but not limited to cellulase(s). Examples of such modifications include modifying endogenous nucleic acid regulatory elements to increase expression of one or more enzymes (e.g., operably linking a gene encoding a target enzyme to a strong promoter), introducing into a microorganism additional copies of endogenous nucleic acid molecules to provide enhanced activity of an enzyme by increasing its production, and operably linking genes encoding one or more enzymes to an inducible promoter or a combination thereof.

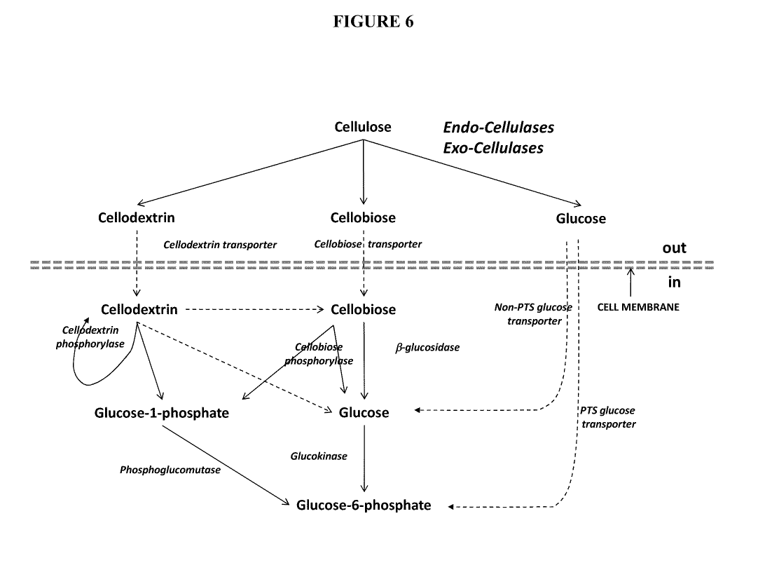

In another embodiment a microorganism can be modified to enhance an activity of one or more cellulases, or enzymes associated with cellulose processing (e.g., FIG. 6). The classification of cellulases is usually based on grouping enzymes together that forms a family with similar or identical activity, but not necessary the same substrate specificity. One of these classifications is the CAZy system (CAZy stands for Carbohydrate-Active enZymes), for example, where there are 115 different Glycoside Hydrolases (GH) listed, named GH1 to GH155. Each of the different protein families usually has a corresponding enzyme activity. This database includes both cellulose and hemicellulase active enzymes. Furthermore, the entire annotated genome of C. phytofermentans is available on the worldwideweb at www.ncbi.nlm.nih.gov/sites/entrez.

Several examples of cellulase enzymes whose function can be enhanced for expression endogenously or for expression heterologously in a microorganism include one or more of the genes disclosed in Table 2.

TABLE 2

| Cellulase | |
|---|---|
| Protein ID | Description (on www.ncbi.nlm.nih.gov/sites/entrez) |
| ABX43556 | Cellulase [Clostridium phytofermentans ISDg] |
| | gi\|160429993\|gb\|ABX43556.1\|[160429993] Cphy_3302 |
| ABX42426 | Cellulase [Clostridium phytofermentans ISDg] |
| | gi\|160428863\|gb\|ABX42426.1\|[160428863] Cphy_2058 |
| ABX41541 | Cellulase [Clostridium phytofermentans ISDg] |
| | gi\|160427978\|gb\|ABX41541.1\|[160427978] Cphy_1163 |
| ABX43720 | Cellulose 1,4-beta-cellobiosidase [Clostridium |
| | phytofermentans ISDg] |
| | gi\|160430157\|gb\|ABX43720.1\|[160430157] Cphy_3367 |
| ABX41478 | Cellulase M Cphy_1100 |
| ABX41884 | Endo-1,4-beta-xylanase Cphy_1510 |
| ABX43721 | Cellulase 1,4-beta-cellobiosidase Cphy_3368 |
| ABX42494 | Mannan endo-1,4-beta-mannosidase, Cellulase 1,4-beta- |
| | cellobiosidase Cphy_2128 |

The Glycosyl hydrolase family 9 (GH9): O-Glycosyl hydrolases are a widespread group of enzymes that hydrolyse the glycosidic bond between two or more carbohydrates, or between a carbohydrate and a non-carbohydrate moiety. A classification system for glycosyl hydrolases, based on sequence similarity, has led to the definition of 85 different families PUBMED:7624375, PUBMED:8535779, PUBMED: This classification is available on the CAZy (CArbohydrate-Active EnZymes) web site PUBMED. Because the fold of proteins is better conserved than their sequences, some of the families can be grouped in ‘clans’. The Glycoside hydrolase family 9 comprises enzymes with several known activities, such as endoglucanase and cellobiohydrolase. In C. phytofermentans, a GH9 cellulase is ABX43720 (Table 2).

Cellulase enzyme activity can be enhanced in a microorganism. In one embodiment a cellulase disclosed in Table 2 is enhanced in a microorganism.

In one embodiment a hydrolytic enzyme is selected from the annotated genome of C. phytofermentans for utilization in a product or process disclosed herein. In one embodiment the hydrolytic enzyme is an endoglucanase, chitinase, cellobiohydrolase or endo-processive cellulases (either on reducing or non-reducing end).

In one embodiment a microorganism, such as C. phytofermentans can be modified to enhance production of one or more cellulase or hydrolase enzymes. In another embodiment one or more enzymes can be heterologous expressed in a host (e.g., a bacteria or yeast). For heterologous expression bacteria or yeast can be modified through recombinant technology. (e.g., Brat et al. Appl. Env. Microbio. 2009; 75(8):2304-2311, disclosing expression of xylose isomerase in S. cerevisiae and which is herein incorporated by reference in its entirety).

In another embodiment other modifications can be made to enhance end-product (e.g., ethanol) production in a recombinant microorganism. For example, the host microorganism can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of ethanol production can be achieved.

A variety of promoters (e.g., constitutive promoters, inducible promoters) can be used to drive expression of the heterologous genes in a recombinant host microorganism.

Promoter elements can be selected and mobilized in a vector (e.g., pIMPCphy). For example, a transcription regulatory sequence is operably linked to gene(s) of interest (e.g., in a expression construct). The promoter can be any array of DNA sequences that interact specifically with cellular transcription factors to regulate transcription of the downstream gene. The selection of a particular promoter depends on what cell type is to be used to express the protein of interest. In one embodiment a transcription regulatory sequences can be derived from the host microorganism. In various embodiments, constitutive or inducible promoters are selected for use in a host cell. Depending on the host cell, there are potentially hundreds of constitutive and inducible promoters which are known and that can be engineered to function in the host cell.

Promoters typically used in recombinant technology, such as E. coli lac and trp operons, the tac promoter, the bacteriophage pL promoter, bacteriophage T7 and SP6 promoters, beta-actin promoter, insulin promoter, baculoviral polyhedrin and p10 promoter, can be used to initiate transcription.

In one embodiment a constitutive promoter can be used including, but not limited to the int promoter of bacteriophage lamda, the bla promoter of the beta-lactamase gene sequence of pBR322, hydA or thlA in Clostridium, S. coelicolor hrdB, or whiE, the CAT promoter of the chloramphenicol acetyl transferase gene sequence of pPR325, Staphylococcal constitutive promoter blaZ and the like.

In another embodiment an inducible promoter can be used that regulates the expression of downstream gene in a controlled manner, such as under a specific condition of a cell culture. Examples of inducible prokaryotic promoters include, but are not limited to, the major right and left promoters of bacteriophage, the trp, reca, lacZ, AraC and gal promoters of E. coli, the alpha-amylase (Ulmanen Ett at., J. Bacteriol. 162:176-182, 1985, which is herein incorporated by reference in its entirety) and the sigma-28-specific promoters of B. subtilis (Gilman et al., Gene sequence 32:11-20 (1984), which is herein incorporated by reference in its entirety), the promoters of the bacteriophages of Bacillus (Gryczan, In: The Molecular Biology of the Bacilli, Academic Press, Inc., NY (1982), which is herein incorporated by reference in its entirety), Streptomyces promoters (Ward et at., Mol. Gen. Genet. 203:468-478, 1986, which is herein incorporated by reference in its entirety), and the like. Exemplary prokaryotic promoters are reviewed by Glick (J. Ind. Microtiot. 1:277-282, 1987, which is herein incorporated by reference in its entirety); Cenatiempo (Biochimie 68:505-516, 1986, which is herein incorporated by reference in its entirety); and Gottesman (Ann. Rev. Genet. 18:415-442, 1984, which is herein incorporated by reference in its entirety).

A promoter that is constitutively active under certain culture conditions, can be inactive in other conditions. For example, the promoter of the hydA gene from Clostridium acetobutylicum, wherein expression is known to be regulated by the environmental pH. Furthermore, temperature-regulated promoters are also known and can be used. In some embodiments, depending on the desired host cell, a pH-regulated or temperature-regulated promoter can be used with an expression constructs to initiate transcription. Other pH-regulatable promoters are known, such as P170 functioning in lactic acid bacteria, as disclosed in US Patent Application No. 20020137140, which is herein incorporated by reference in its entirety.

In general, to express the desired gene/nucleotide sequence efficiently, various promoters can be used; e.g., the original promoter of the gene, promoters of antibiotic resistance genes such as for instance kanamycin resistant gene of Tn5, ampicillin resistant gene of pBR322, and promoters of lambda phage and any promoters which can be functional in the host cell. For expression, other regulatory elements, such as for instance a Shine-Dalgarno (SD) sequence (e.g., AGGAGG and so on including natural and synthetic sequences operable in a host cell) and a transcriptional terminator (inverted repeat structure including any natural and synthetic sequence) which are operable in a host cell (into which a coding sequence is introduced to provide a recombinant cell) can be used with the above described promoters.

Examples of promoters that can be used with a product or process disclosed herein include those disclosed in the following patent documents: US20040171824, U.S. Pat. No. 6,410,317, WO 2005/024019, which are herein incorporated by reference in their entirety. Several promoter-operator systems, such as lac, (D. V. Goeddel et al., “Expression in Escherichia coli of Chemically Synthesized Genes for Human Insulin”, Proc. Nat. Acad. Sci. U.S.A., 76:106-110 (1979), which is herein incorporated by reference in its entirety); trp (J. D. Windass et al. “The Construction of a Synthetic Escherichia coli Trp Promoter and Its Use in the Expression of a Synthetic Interferon Gene”, Nucl. Acids. Res., 10:6639-57 (1982), which is herein incorporated by reference in its entirety) and λ PL operons (R. Crowl et al., “Versatile Expression Vectors for High-Level Synthesis of Cloned Gene Products in Escherichia coli”, Gene, 38:31-38 (1985), which is herein incorporated by reference in its entirety) in E. coli and have been used for the regulation of gene expression in recombinant cells. The corresponding repressors are the lac repressor, trpR and cI, respectively.

Repressors are protein molecules that bind specifically to particular operators. For example, the lac repressor molecule binds to the operator of the lac promoter-operator system, while the cro repressor binds to the operator of the lambda pR promoter. Other combinations of repressor and operator are known in the art. See, e.g., J. D. Watson et al., Molecular Biology Of The Gene, p. 373 (4th ed. 1987), which is herein incorporated by reference in its entirety. The structure formed by the repressor and operator blocks the productive interaction of the associated promoter with RNA polymerase, thereby preventing transcription. Other molecules, termed inducers, bind to repressors, thereby preventing the repressor from binding to its operator. Thus, the suppression of protein expression by repressor molecules can be reversed by reducing the concentration of repressor (depression) or by neutralizing the repressor with an inducer.

Analogous promoter-operator systems and inducers are known in other microorganisms. In yeast, the GAL10 and GAL1 promoters are repressed by extracellular glucose, and activated by addition of galactose, an inducer. Protein GAL80 is a repressor for the system, and GAL4 is a transcriptional activator. Binding of GAL80 to galactose prevents GAL80 from binding GAL4. Then, GAL4 can bind to an upstream activation sequence (UAS) activating transcription. See Y. Oshima, “Regulatory Circuits For Gene Expression: The Metabolisms Of Galactose And Phosphate” in The Molecular Biology Of The Yeast Sacharomyces, Metabolism And Gene Expression, J. N. Strathern et al. eds. (1982), which are herein incorporated by reference in their entirety.

Transcription under the control of the PHO5 promoter is repressed by extracellular inorganic phosphate, and induced to a high level when phosphate is depleted. R. A. Kramer and N. Andersen, “Isolation of Yeast Genes With mRNA Levels Controlled By Phosphate Concentration”, Proc. Nat. Acad. Sci. U.S.A., 77:6451-6545 (1980), which is herein incorporated by reference in its entirety. A number of regulatory genes for PHO5 expression have been identified, including some involved in phosphate regulation.

Matα2 is a temperature-regulated promoter system in yeast. A repressor protein, operator and promoter sites have been identified in this system. A. Z. Sledziewski et al., “Construction Of Temperature-Regulated Yeast Promoters Using The Matα2 Repression System”, Bio/Technology, 6:411-16 (1988), which is herein incorporated by reference in its entirety.

Another example of a repressor system in yeast is the CUP1 promoter, which can be induced by Cu⁺2 ions. The CUP1 promoter is regulated by a metallothionine protein. J. A. Gorman et al., “Regulation Of The Yeast Metallothionine Gene”, Gene, 48:13-22 (1986), which is herein incorporated by reference in its entirety.

Similarly, to obtain a desired expression level of one or more cellulases, a higher copy number plasmid can be used. Constructs can be prepared for chromosomal integration of the desired genes. Chromosomal integration of foreign genes can offer several advantages over plasmid-based constructions. Ethanologenic genes have been integrated chromosomally in E. coli B; see Ohta et al. (1991) Appl. Environ. Microbiol. 57:893-900, which is herein incorporated by reference in its entirety. In general, this is accomplished by purification of a DNA fragment containing (1) the desired genes upstream from an antibiotic resistance gene and (2) a fragment of homologous DNA from the target microorganism. This DNA can be ligated to form circles without replicons and used for transformation. Thus, the gene of interest can be introduced in a heterologous host such as E. coli, and short, random fragments can be isolated and operably linked to target genes (e.g., genes encoding cellulase enzymes) to promote homologous recombination.

In some embodiments, a microorganism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized microorganisms on substrates that comprise carbon sources that will be used during the generation of end-products by fermentation. Screening can also include measuring the production of end-products during growth of the microorganism, or measuring the digestion or assimilation of the carbon source(s). The isolates so obtained can further be transformed with recombinant polynucleotides or used in combination with any of the methods and compositions provided herein to further enhance biofuel production.

In some embodiments host cells (e.g., microorganisms) can be transformed with multiple genes encoding one or more enzymes. For example, a single transformed cell can contain exogenous nucleic acids encoding an entire biodegradation pathway. One example of a pathway can include genes encoding an exo-β-glucanase, and endo-β-glucanase, and an endoxylanase. Such cells transformed with entire pathways and/or enzymes extracted from them, can saccharify certain components of biomass more rapidly than the naturally-occurring organism. Constructs can contain multiple copies of the same gene, and/or multiple genes encoding the same enzyme from different organisms, and/or multiple genes with mutations in one or more parts of the coding sequences. For example, multiple copies of Cphy_{—}3367 or Cphy_{—}3368 (SEQ ID NO:5 or SEQ ID NO:8, respectively) can increase saccharification, thus increasing the rate and yield of fermentation products. In some embodiments, the nucleic acid sequences encoding the genes can be similar or identical to the endogenous gene. There can be a percent similarity of 70% or more in comparing the base pairs of the sequences.

In another embodiment, more effective biomass degradation pathways can be created by transforming host cells with multiple copies of enzymes of the pathway and then combining the cells producing the individual enzymes. This approach allows for the combination of enzymes to more particularly match the biomass of interest by altering the relative ratios of the multiple-transformed strains. In one embodiment two times as many cells expressing the first enzyme of a pathway can be added to a mix where the first step of the reaction pathway is a limiting step of the overall reaction pathway.

In one embodiment biomass-degrading enzymes are made by transforming host cells (e.g., microbial cells such as bacteria, especially Clostridial cells, algae, and fungi) and/or organisms comprising host cells with nucleic acids encoding one or more different biomass degrading enzymes (e.g., cellulolytic enzymes, hemicellulolytic enzymes, xylanases, lignases and cellulases). In some embodiments, a single enzyme can be produced. For example, a cellulase which breaks down pretreated cellulose fragments into cellodextrins or double glucose molecules (cellobiose) or a cellulase which splits cellobiose into glucose, can be produced. In other embodiments, multiple copies of an enzyme can be transformed into an organism to overcome a rate-limiting step of a reaction pathway.

### EXAMPLES

### Example 1

### Cellulase Enzyme Addition

To study the effects of exogenous enzyme supplementation, hydrolytic enzyme mixtures and individual hydrolytic enzymes were added during the fermentation of a corn stover biomass.

The following operating conditions and process parameters for C. phytofermentans were followed for fermentation of NaOH-pretreated corn stover with enzyme augmentation in 250 ml shake flasks with 100 ml of culture medium (Table 3).

TABLE 3
Operating conditions

| | |
|---|---|
| pH | 6.5; range of from about 6.0 to about 7.0 |
| Temperature | 35° C. . . . Shake flasks were incubated in |
| | temperature controlled cabinets |
| Agitation | 175 rpm |
| Degassing | Sparging with N₂ to achieve redox potential less |
| | than −300 mV |
| Base for pH control | 4N NaOH |
| Mode of operation | Batch |
| Inoculation size | 0.5 g/L on dry wt. (2 × 10⁹ CFU) use dfo reach |
| | flask |

Seed propagation media (QM1) recipe:

| | g/L: |
|---|---|
| QM Base Media: | |
| KH₂PO₄ | 1.92 |
| K₂HPO₄ | 10.60 |
| Ammonium sulfate | 4.60 |
| Sodium citrate tribasic * 2H₂O | 3.00 |
| Bacto yeast extract | 6.00 |
| Cysteine | 2.00 |
| 20x Substrate Stock | |
| Maltose | 400.00 |
| 100X QM Salts solution: | |
| MgCl₂•6H₂O | 100 |
| CaCl₂•2H₂O | 15 |
| FeSO₄•7H₂O | 0.125 |

The seed propagation media was prepared according to the recipe above. Base media, salts and substrates were degassed with nitrogen prior to autoclave sterilization. Following sterilization, 94 ml of base media was combined with 1 ml of 100× salts and 5 mls of 20× substrate to achieve a final concentrations. All additions were prepared anaerobically and aseptically.

Fermentation Media: (FM Media)

Base media (g/L) was prepared with: 50 g/l NaOH pretreated corn stover, yeast extract 10, corn steep powder 5, K₂HPO₄ 3, KH₂PO₄ 1.6, TriSodium citrate 2H₂O₂ 2, Citric acid H₂O 1.2, (NH₄)₂SO₄ 0.5, NaCl 1, Cysteine.HCl 1, dissolved in deionized water to achieve final volume, adjusted to pH to 6.5, degassed with nitrogen and autoclaved 121° C. for 30 min.

100× Salt Stock (g/L):

MgCl₂.6H₂O 80, CaCl₂.2H₂O 10, FeSO₄.7H₂O 0.125, TriSodium citrate.2H₂O₂ 3.0

Culturing Procedure:

The fermentation media was prepared according to the protocol above. Components of the Base media were combined to a single vessel and degassed with nitrogen prior to sterilization. A 100× salts stock was prepared and sterilized separately. After sterilization base media was supplemented with a 1% v/v dose of 100× salts to achieve a final concentration. All additions were prepared anaerobically and aseptically.

Enzymes were obtained from Novozymes and mixtures (cocktails) were prepared separately and sterilized by sterile filtration using 0.2 μm filters. The prepared enzymes were then added to the FM corn stover media immediately prior to time of inoculation. Other enzymes and mixtures of enzymes from several different manufacturers were also tested with similar results.

Inoculum of Clostridium phytofermentans was prepared by propagation in QM media 24 hrs to an active cell density of 2×10⁹ cells per ml. The cells were concentrated by centrifugation and then transferred into the FM media bottles to achieve an initial cell density of 2×10⁹ cells per ml for the start of fermentation.

Cultures were then incubated at pH 6.5 and at 35° C. for 120 hr or until fermentations were complete. Product formation was determined by HPLC analysis using refractive index detection. Compositional analysis for the NaOH-treated corn stover was obtained via NREL standard methods using two-stage acid hydrolysis procedures.

The addition of cellulase mixtures exhibiting as little as 0.4 FPU per gram of glucan supplemented with B-glucosidase, hemicellulase, pectinase and xylanase resulted in >95% theoretical yields of ethanol from fermentation of 50 g/l NaOH-treated corn stover. The addition of a single endo-cellulase complex at 1.68FPU per gram of glucan resulted in greater than 90% theoretical saccharification and greater than 70% fermentation yield. (Table 4.) Addition of B-glucosidase, xylanase, or hemicellulase alone had little to no impact to the rate, titer or yield of Q fermentations. Based on the microorganism's metabolism of cellulose in the conditions studied, these enzymes were not observed to impact the fermentation process. For fermentation, addition of B-glucosidase does not significantly impact the fermentation process.

The table below shows the adjusted loadings in terms of FPU/gram of glucan as a standard enzyme unit. The activities were adjusted based on the reduction in the enzyme-reduced activity at the lowered temperature and higher pH ranges. Experiments were performed at pH 6.5 and temperature of 35° C., which resulted in more than 80% loss of cellulase activity. This factor was figured into the loading calculations—FPU: filter paper unit, CBU cellobiosidase unit, FBG fungal glucanase unit, FXU: fungal xylanase unit.

TABLE 4

| | *FPU/ | CBU/ | *FBG/ | *FXU/ |
|---|---|---|---|---|
| Description of | gram | gram | gram | gram |
| enzyme cocktail | glucan | glucan | biomass | biomass |
| 2x mixture | 3.36 | 15.00 | 2.36 | 19.3 |
| 1x mixture | 1.68 | 7.50 | 1.18 | 9.65 |
| .5x mixture | 0.84 | 3.75 | 0.59 | 4.83 |
| .25x mixture | 0.42 | 1.88 | 0.30 | 2.41 |
| NS50013 cellulase | 1.68 | NA | NA | NA |
| NS50010 B-glucosidase | NA | 7.5 | NA | NA |
| NS50012 hemicellulase | NA | NA | 0.28 | NA |
| NS50030 xylanase | NA | NA | 0.25 | NA |
| NS22002 glucanase/xylanase | NA | NA | 0.9 | 9.4 |
| No exogenous enzyme | NA | NA | NA | NA |

Compositional analysis of 1% NaOH treated corn stover
*activity adjusted based on % activity retained at 35° C.

Carbohydrate analysis for NaOH-treated corn stover; Glucan: 53.37%; Xylan: 27.5%; Arabinan: 3.6%. Total sugar equivalents: 95.4%.

The difference in the fermentation profiles for the cultures with enzyme mixture additions from 0.25 to 2× result in the theoretical ethanol conversions described in FIG. 1A. The curves for individual enzyme augmentations illustrate the impact of cellulase additions and also clearly demonstrate the ineffectiveness of B-glucosidase, xylanase and hemicelluloses additions described in FIG. 1B.

Conversion efficiency of SSF fermentations for Corn stover with various enzyme loadings and individual enzyme loadings is further provided in Table 5.

TABLE 5

| | | Initial | | | consumed | | |
|---|---|---|---|---|---|---|---|
| | | sugar | Residual | | sugars | Ethanol | |
| Description of | Time | equivalents | sugars | Ethanol | (assumes | yield (% | Saccharification |
| enzyme cocktail | (days) | g/l | g/l | titer g/l | .45 g/g) | theoretical) | yield |
| 2x mixture | 7.00 | 48.66 | 3.90 | 23.66 | 52.58 | 95% | 116% |
| 1x mixture | 7.00 | 48.66 | 2.32 | 22.90 | 50.89 | 92% | 109% |
| .5x mixture | 7.00 | 48.66 | 0.72 | 23.83 | 52.96 | 96% | 110% |
| .25x mixture | 7.00 | 48.66 | 1.12 | 22.53 | 50.07 | 91% | 105% |
| NS50013 | 7.00 | 48.66 | 5.66 | 17.60 | 39.11 | 71% | 92% |
| Cellulase | | | | | | | |
| NS50010 B- | 7.00 | 48.66 | 2.65 | 6.88 | 15.29 | 28% | 37% |
| glucosidase | | | | | | | |
| NS50012 | 7.00 | 48.66 | 3.20 | 6.91 | 15.36 | 28% | 38% |
| Hemicellulase | | | | | | | |
| NS50030 xylanase | 7.00 | 48.66 | 1.66 | 7.88 | 17.51 | 32% | 39% |
| NS22002 | 7.00 | 48.66 | 2.73 | 10.35 | 23.00 | 42% | 53% |
| glucanase/xylanase | | | | | | | |
| No exogenous | 7.00 | 48.66 | 2.40 | 6.13 | 13.62 | 25% | 33% |
| enzyme | | | | | | | |

| | glucan | xylan | arabinan | Total Sugar equivalents |
|---|---|---|---|---|
| Compositional analysis of | 53.3% | 27.4% | 3.6% | 95.4% |
| 1% NaOH treated corn | | | | |
| stover | | | | |

*activity adjusted based on % activity retained at 35° C.

FIG. 2 and FIG. 4 show the synergistic effect of hydrolytic enzyme on C. phytofermentans saccharification efficiency. FIG. 3A demonstrates the reduced amount of enzyme mixture necessary for peak ethanol yield during C. phytofermentans saccharification and fermentation of corn stover. FIG. 3B shows that cellulase alone is the hydrolytic enzyme responsible for the higher ethanol yield during C. phytofermentans saccharification and fermentation of corn stover. During these fermentations, significantly lower amounts of hydrolytic enzymes than normally used during biofuel production with other organisms resulted in high rates and yield of ethanol with C. phytofermentans (FIG. 5).

### Example 2

### Microorganism Modification

Constitutive Expression of Cellulases I

pIMPCphy

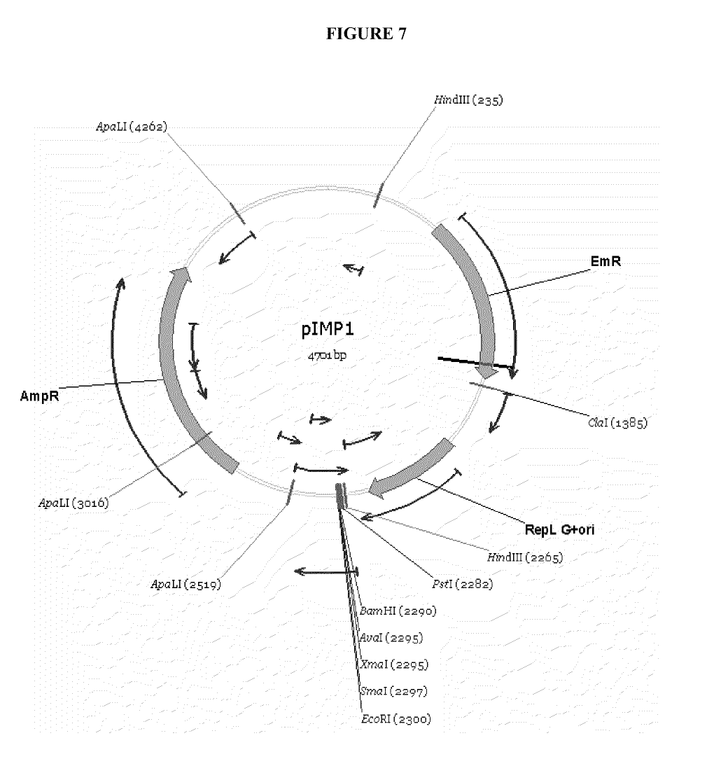

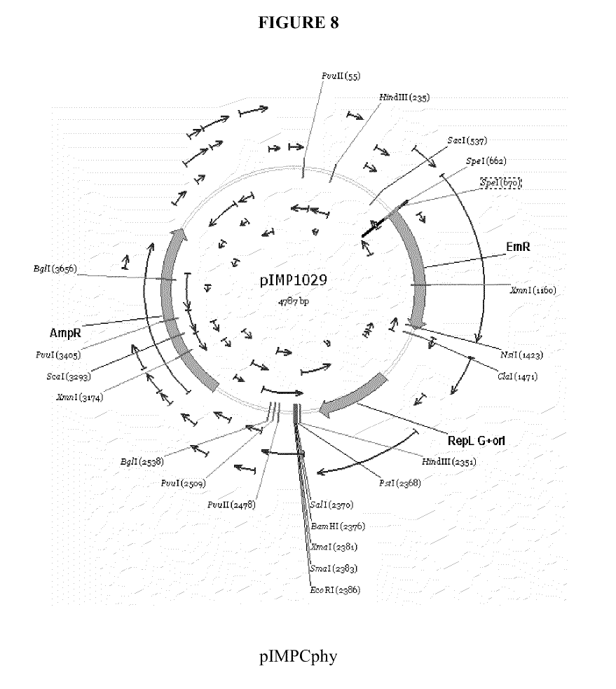

Plasmids suitable for use in Clostridium phytofermentans were constructed using portions of plasmids obtained from bacterial culture collections (Deutsche Sammlung von Mikroorganismen and Zellkulturen GmbH, Inhoffenstraβe 7 B, 38124 Braunschweig, Germany, hereinafter “DSMZ”). Plasmid pIMP1 is a non-conjugal shuttle vector that can replicate in Escherichia coli and C. phytofermentans; additionally, pIMP1 (FIG. 7) encodes for resistance to erythromycin (Em^{R}). The origin of transfer for the RK2 conjugal system was obtained from plasmid pRK29O (DSMZ) as DSM 3928, and the other conjugation functions of RK2 were obtained from pRK2013 (DSMZ) as DSM 5599. The polymerase chain reaction (PCR) was used to amplify the 112 base pair origin of transfer region (oriT) from pRK290 using primers that added ClaI restriction sites flanking the oriT region. This DNA fragment was inserted into the ClaI site on pIMP1 to yield plasmid pIMPT. pIMPT was shown to able to be transferred from one strain of E. coli to another when pRK2013 was also present to supply other conjugation functions. PCR was used to amplify the promoter of the alcohol dehydrogenase (Adh) gene Cphy_{—}1029 from the C. phytofermentans chromosome and it was used to replace the promoter of the erythromycin gene in pIMPT to create pIMPTCphy (FIG. 8). The successful transfer of pIMPTCphy into C. phytofermentans via electroporation was demonstrated by the ability to grow in the presence of 10 μg/mL erythromycin. In addition to phenotypic proof of electroporation provided by the growth on erythromycin, successive plasmid isolations from C. phytofermentans confirmed that the same plasmid was isolated from Clostridium phytofermentans and transferred into E. coli and recovered.

The method of conjugal transfer of pIMPTCphy from E. coli to C. phytofermentans involved constructing an E. coli strain (DHSalpha) that contains both pIMPTCphy and pRK2013. Fresh cells E. coli culture and fresh cells of the C. phytofermentans recipient culture were obtained by growth to mid-log phase using appropriate growth media (L broth and QM1 media respectively). The two bacterial cultures were then centrifuged to yield cell pellets and the pellets resuspended in the same media to obtain cell suspensions that were concentrated about ten-fold having cell densities of about 10¹⁰ cells per ml. These concentrated cell suspensions were then mixed to achieve a donor-to-recipient ratio of five-to-one, then the cell suspension was spotted onto QM1 agar plates and incubated anaerobically at 30° C. for 24 hours. The cell mixture was removed from the QM1 plate and placed on solid or in liquid QM1 media containing antibiotics that allow the survival of C. phytofermentans recipient cells expressing erythromycin resistance. This was accomplished by using a combination of antibiotics consisting of trimethoprim (20 μg/ml), cycloserine (250 μg/ml), and erythromycin (10 μg/ml). The E. coli donor was unable to survive exposure to these concentrations of trimethoprim and cycloserine, while the C. phytofermentans recipient was unable to survive exposure to this concentration of erythromycin (but could tolerate trimethoprim and cycloserine at these concentrations). Accordingly, after anaerobic incubation on antibiotic-containing plates or liquid media for 5 to 7 days at 30° C., derivatives of C. phytofermentans were obtained that were erythromycin resistant and these C. phytofermentans derivatives were subsequently shown to contain pIMPCphy as demonstrated by PCR analyses.

A map of the plasmid pIMPCphy is shown in FIG. 8, and the DNA sequence of this plasmid is provided as SEQ ID NO:1.

SEQ ID NO: 1:
gcgcccaatacgcaaaccgcctctccccgcgcgttggccgattcattaat
gcagctggcacgacaggtttcccgactggaaagcgggcagtgagcgcaac
gcaattaatgtgagttagctcactcattaggcaccccaggctttacactt
tatgcttccggctcgtatgttgtgtggaattgtgagcggataacaatttc
acacaggaaacagctatgaccatgattacgccaaagctttggctaacaca
cacgccattccaaccaatagttttctcggcataaagccatgctctgacgc
ttaaatgcactaatgccttaaaaaaacattaaagtctaacacactagact
tatttacttcgtaattaagtcgttaaaccgtgtgctctacgaccaaaagt
ataaaacctttaagaactttcttttttcttgtaaaaaaagaaactagata
aatctctcatatcttttattcaataatcgcatcagattgcagtataaatt
taacgatcactcatcatgttcatatttatcagagctccttatattttatt
tcgatttatttgttatttatttaacatttttctattgacctcatcttttc
tatgtgttattcttttgttaattgtttacaaataatctacgatacataga
aggaggaaaaactagtatactagtatgaacgagaaaaatataaaacacag
tcaaaactttattacttcaaaacataatatagataaaataatgacaaata
taagattaaatgaacatgataatatctttgaaatcggctcaggaaaaggg
cattttacccttgaattagtacagaggtgtaatttcgtaactgccattga
aatagaccataaattatgcaaaactacagaaaataaacttgttgatcacg
ataatttccaagttttaaacaaggatatattgcagtttaaatttcctaaa
aaccaatcctataaaatatttggtaatataccttataacataagtacgga
tataatacgcaaaattgtttttgatagtatagctgatgagatttatttaa
tcgtggaatacgggtttgctaaaagattattaaatacaaaacgctcattg
gcattatttttaatggcagaagttgatatttctatattaagtatggttcc
aagagaatattttcatcctaaacctaaagtgaatagctcacttatcagat
taaatagaaaaaaatcaagaatatcacacaaagataaacagaagtataat
tatttcgttatgaaatgggttaacaaagaatacaagaaaatatttacaaa
aaatcaatttaacaattccttaaaacatgcaggaattgacgatttaaaca
atattagctttgaacaattcttatctcttttcaatagctataaattattt
aataagtaagttaagggatgcataaactgcatcccttaacttgtttttcg
tgtacctattttttgtgaatcgatccggccagcctcgcagagcaggattc
ccgttgagcaccgccaggtgcgaataagggacagtgaagaaggaacaccc
gctcgcgggtgggcctacttcacctatcctgcccggatcgattatgtctt
ttgcgcattcacttcttttctatataaatatgagcgaagcgaataagcgt
cggaaaagcagcaaaaagtttcctttttgctgttggagcatgggggttca
gggggtgcagtatctgacgtcaatgccgagcgaaagcgagccgaagggta
gcatttacgttagataaccccctgatatgctccgacgctttatatagaaa
agaagattcaactaggtaaaatcttaatataggttgagatgataaggttt
ataaggaatttgtttgttctaatttttcactcattttgttctaatttctt
ttaacaaatgttcttttttttttagaacagttatgatatagttagaatag
tttaaaataaggagtgagaaaaagatgaaagaaagatatggaacagtcta
taaaggctctcagaggctcatagacgaagaaagtggagaagtcatagagg
tagacaagttataccgtaaacaaacgtctggtaacttcgtaaaggcatat
atagtgcaattaataagtatgttagatatgattggcggaaaaaaacttaa
aatcgttaactatatcctagataatgtccacttaagtaacaatacaatga
tagctacaacaagagaaatagcaaaagctacaggaacaagtctacaaaca
gtaataacaacacttaaaatcttagaagaaggaaatattataaaaagaaa
aactggagtattaatgttaaaccctgaactactaatgagaggcgacgacc
aaaaacaaaaatacctcttactcgaatttgggaactttgagcaagaggca
aatgaaatagattgacctcccaataacaccacgtagttattgggaggtca
atctatgaaatgcgattaagcttagcttggctgcaggtcgacggatcccc
gggaattcactggccgtcgttttacaacgtcgtgactgggaaaaccctgg
cgttacccaacttaatcgccttgcagcacatccccctttcgccagctggc
gtaatagcgaagaggcccgcaccgatcgcccttcccaacagttgcgcagc
ctgaatggcgaatggcgcctgatgcggtattttctccttacgcatctgtg
cggtatttcacaccgcatatggtgcactctcagtacaatctgctctgatg
ccgcatagttaagccagccccgacacccgccaacacccgctgacgcgccc
tgacgggcttgtctgctcccggcatccgcttacagacaagctgtgaccgt
ctccgggagctgcatgtgtcagaggttttcaccgtcatcaccgaaacgcg
cgagacgaaagggcctcgtgatacgcctatttttataggttaatgtcatg
ataataatggtttcttagacgtcaggtggcacttttcggggaaatgtgcg
cggaacccctatttgtttatttttctaaatacattcaaatatgtatccgc
tcatgagacaataaccctgataaatgcttcaataatattgaaaaaggaag
agtatgagtattcaacatttccgtgtcgcccttattcccttttttgcggc
attttgccttcctgtttttgctcacccagaaacgctggtgaaagtaaaag
atgctgaagatcagttgggtgcacgagtgggttacatcgaactggatctc
aacagcggtaagatccttgagagttttcgccccgaagaacgttttccaat
gatgagcacttttaaagttctgctatgtggcgcggtattatcccgtattg
acgccgggcaagagcaactcggtcgccgcatacactattctcagaatgac
ttggttgagtactcaccagtcacagaaaagcatcttacggatggcatgac
agtaagagaattatgcagtgctgccataaccatgagtgataacactgcgg
ccaacttacttctgacaacgatcggaggaccgaaggagctaaccgctttt
ttgcacaacatgggggatcatgtaactcgccttgatcgttgggaaccgga
gctgaatgaagccataccaaacgacgagcgtgacaccacgatgcctgtag
caatggcaacaacgttgcgcaaactattaactggcgaactacttactcta
gcttcccggcaacaattaatagactggatggaggcggataaagttgcagg
accacttctgcgctcggcccttccggctggctggtttattgctgataaat
ctggagccggtgagcgtgggtctcgcggtatcattgcagcactggggcca
gatggtaagccctcccgtatcgtagttatctacacgacggggagtcaggc
aactatggatgaacgaaatagacagatcgctgagataggtgcctcactga
ttaagcattggtaactgtcagaccaagtttactcatatatactttagatt
gatttaaaacttcatttttaatttaaaaggatctaggtgaagatcctttt
tgataatctcatgaccaaaatcccttaacgtgagttttcgttccactgag
cgtcagaccccgtagaaaagatcaaaggatcttcttgagatccttttttt
ctgcgcgtaatctgctgcttgcaaacaaaaaaaccaccgctaccagcggt
ggtttgtttgccggatcaagagctaccaactctttttccgaaggtaactg
gcttcagcagagcgcagataccaaatactgtccttctagtgtagccgtag
ttaggccaccacttcaagaactctgtagcaccgcctacatacctcgctct
gctaatcctgttaccagtggctgctgccagtggcgataagtcgtgtctta
ccgggttggactcaagacgatagttaccggataaggcgcagcggtcgggc
tgaacggggggttcgtgcacacagcccagcttggagcgaacgacctacac
cgaactgagatacctacagcgtgagctatgagaaagcgccacgcttcccg
aagggagaaaggcggacaggtatccggtaagcggcagggtcggaacagga
gagcgcacgagggagcttccagggggaaacgcctggtatctttatagtcc
tgtcgggtttcgccacctctgacttgagcgtcgatttttgtgatgctcgt
caggggggcggagcctatggaaaaacgccagcaacgcggcctttttacgg
ttcctggccttttgctggccttttgctcacatgttctttcctgcgttatc
ccctgattctgtggataaccgtattaccgcctttgagtgagctgataccg
ctcgccgcagccgaacgccgagcgcagcgagtcagtgagcgaggaagcgg
aaga.

The vector pIMPCphy was constructed as a shuttle vector for C. phytofermentans. It has an Ampicillin-resistance cassette and an Origin of Replication (ori) for selection and replication in E. coli. It contains a Gram-positive origin of replication that allows the replication of the plasmid in C. phytofermentans. In order to select for the presence of the plasmid, the pIMPCphy carries an erythromycin resistance gene under the control of the C. phytofermentans promoter of the gene Cphy1029. This plasmid can be transferred to C. phytofermentans by electroporation or by transconjugation with an E. coli strain that has a mobilizing plasmid, for example pRK2030. A plasmid map of pIMPCphy is depicted in FIG. 8. pIMPCphy is an effective replicative vector system for all microbes, including all gram⁺ and gram⁻ bacteria, and fungi (including yeasts).

Constitutive Promoter

In a first step, several promoters from C. phytofermentans were chosen that show high expression of their corresponding genes in all growth stages as well as on different substrates. A promoter element can be selected by selecting key genes that would necessarily be involved in constitutive pathways (e.g., ribosomal genes, or for ethanol production, alcohol dehydrogenase genes). Examples of promoters from such genes include but are not limited to:

Cphy_{—}1029: iron-containing alcohol dehydrogenase

Cphy_{—}3510: Ig domain-containing protein

Cphy_{—}3925: bifunctional acetaldehyde-CoA/alcohol dehydrogenase

Cloning of Promoter

The different promoters in the upstream regions of the genes were amplified by PCR. The primers for this PCR reaction were chosen in a way that they include the promoter region but do not include the ribosome binding sites of the downstream gene. The primers were engineered to introduce restriction sites at the end of the promoter fragments that are present in the multiple cloning site of pIMPCphy but are otherwise not present in the promoter region itself, for example SalI, BamHI, XmaI, SmaI, EcoRI.

The PCR reaction was performed with a commercially available PCR Kit, e.g. GoTaq® Green Master Mix (Promega Corporation, 2800 Woods Hollow Road, Madison, Wis. 53711 USA), according to the manufacturer's conditions. The reaction is run in a thermal cycler, e.g. Gene Amp System 2400 (PerkinElmer, 940 Winter St., Waltham Mass. 02451 USA). The PCR products were purified with the GenElute™ PCR Clean-Up Kit (Sigma-Aldrich Corp., St. Louis, Mo., USA). Both the purified PCR products as well as the plasmid pIMPCphy were then digested with the corresponding enzymes with the appropriate amounts according to the manufacturer's conditions (restriction enzymes from New England Biolabs, 240 County Road, Ipswich, Mass. 01938 USA and Promega). The PCR products and the plasmid were then analyzed and gel-purified on a Recovery FlashGel (Lonza Biologics, Inc., 101 International Drive, Portsmouth, N.H. 03801 USA). The PCR products were subsequently ligated to the plasmid with the Quick Ligation Kit (New England Biolabs) and competent cells of E. coli (DH5α) are transformed with the ligation mixtures and plated on LB plates with 100 μg/ml ampicillin. The plates are incubated overnight at 37° C.

Ampicillin resistant E. coli colonies were picked from the plates and restreaked on new selective plates. After growth at 37° C., liquid LB medium with 100 μg/ml ampicillin was inoculated with a single colony and grown overnight at 37° C. Plasmids were isolated from the liquid culture with the Gene Elute™ Plasmid isolation kit.

Miniprep Kit (Sigma-Aldrich).

Plasmids were checked for the right insert by PCR reaction and restriction digest with the appropriate primers and by restriction enzymes respectively. To ensure the sequence integrity, the insert is sequenced at this step.

Cloning of Cellulase Genes

One or more genes disclosed in Table 2, which can include each gene's own ribosome binding sites, were amplified via PCR and subsequently digested with the appropriate enzymes as described previously under Cloning of Promoter. Resulting plasmids were also treated with the corresponding restriction enzymes and the amplified genes are mobilized into plasmids through standard ligation. E. coli were transformed with the plasmids and correct inserts were verified from transformants selected on selection plates.

Transconjugation

E. coli DH5α along with the helper plasmid pRK2030, were transformed with the different plasmids discussed above. E. coli colonies with both of the foregoing plasmids were selected on LB plates with 100 μg/ml ampicillin and 50 μg/ml kanamycin after growing overnight at 37° C. Single colonies were obtained after re-streaking on selective plates at 37° C. Growth media for E. coli (e.g. LB or LB supplemented with 1% glucose and 1% cellobiose) was inoculated with a single colony and either grown aerobically at 37° C. or anaerobically at 35° C. overnight. Fresh growth media was inoculated 1:100 with the overnight culture and grown until mid log phase. A C. phytofermentans strain was also grown in the same media until mid log.

The two different cultures, C. phytofermentans and E. coli with pRK2030 and one of the plasmids, were then mixed in different ratios, e.g. 1:1000, 1:100, 1:10, 1:1, 10:1, 100:1, 1000:1. The mating was performed in either liquid media, on plates or on 25 mm Nucleopore Track-Etch Membrane (Whatman, Inc., 800 Centennial Avenue, Piscataway, N.J. 08854 USA) at 35° C. The time was varied between 2 h and 24 h, and the mating media was the same growth media in which the culture was grown prior to the mating. After the mating procedure, the bacteria mixture was either spread directly onto plates or first grown on liquid media for 6 h to 18 h and then plated. The plates contain 10 μg/ml erythromycin as selective agent for C. phytofermentans and 10 μg/ml Trimethoprim, 150 μg/ml Cyclosporin and 100 μ/ml Nalidixic acid as counter selectable media for E. coli.

After 3 to 5 days incubation at 35° C., erythromycin-resistant colonies were picked from the plates and restreaked on fresh selective plates. Single colonies were picked and the presence of the plasmid is confirmed by PCR reaction.

Cellulase Gene Expression

The expression of the cellulase genes on the different plasmids was then tested under conditions where there is little to no expression of the corresponding genes from the chromosomal locus. Positive candidates showed constitutive expression of the cloned cellulases.

Constitutive Expression of Cellulases I

pCphyP3510-1163

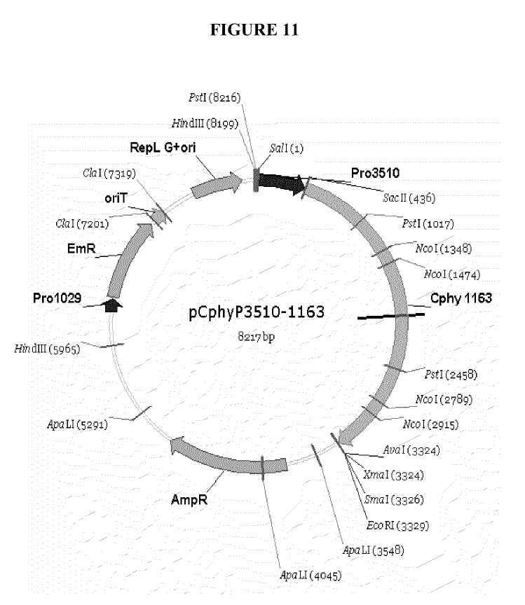

Two primers were chosen to amplify Cphy_{—}1163 using C. phytofermentans genomic DNA as template. The two primers were: cphy_{—}1163F: 5′-CCG CGG AGG AGG GTT TTG TAT GAG TAA AAT CAG AAG AAT AGT TTC-3 (SEQ ID NO: 3), which contained a SacII restriction enzyme site and ribosomal site; and cphy_{—}1163R: CCC GGG TTA GTG GTG GTG GTG GTG GTG TTT TCC ATA ATA TTG CCC TAA TGA (SEQ ID NO: 4), which containing a XmaI site and His-tag (SEQ ID NO: 31). The amplified gene was cloned into Topo-TA first, then digested with SacII and XmaI, the cphy_{—}1163 fragment was gel purified and ligated with pCPHY3510 digested with SacII and XmaI, respectively. The plasmid was transformed into E. coli, purified and then transformed into C. phytofermentans by electroporation. The plasmid map is shown in FIG. 11.

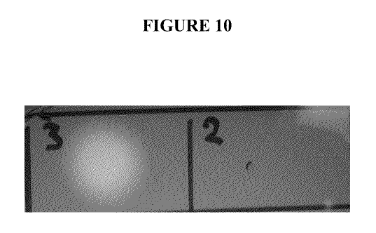

The transformants from the QM plate, which contained 20 μg/ml of erythromycin, were transformed into QM liquid medium, which contained 2% cellobiose and 20 μg/ml of erythromycin. The enzyme activities from the supernatant of overnight culture were assayed by CMC-congo red plate assay and Cellazyme T assay kit (Megazyme International Ireland, Ltd., Bray Business Park, Bray, Co., Wicklow, Ireland). The CMC-congo plate and the Cellazyme T assays indicated the transformant of C. phytofermentans/pCphy3510_{—}1163 showed increased activity than that of the control strain (FIG. 10). The CEL-T assay showed the tranformant had an activity level of 54.5 mU/ml (left box “3”) whereas the control activity was only 3.7 mU/ml (right box “2”).

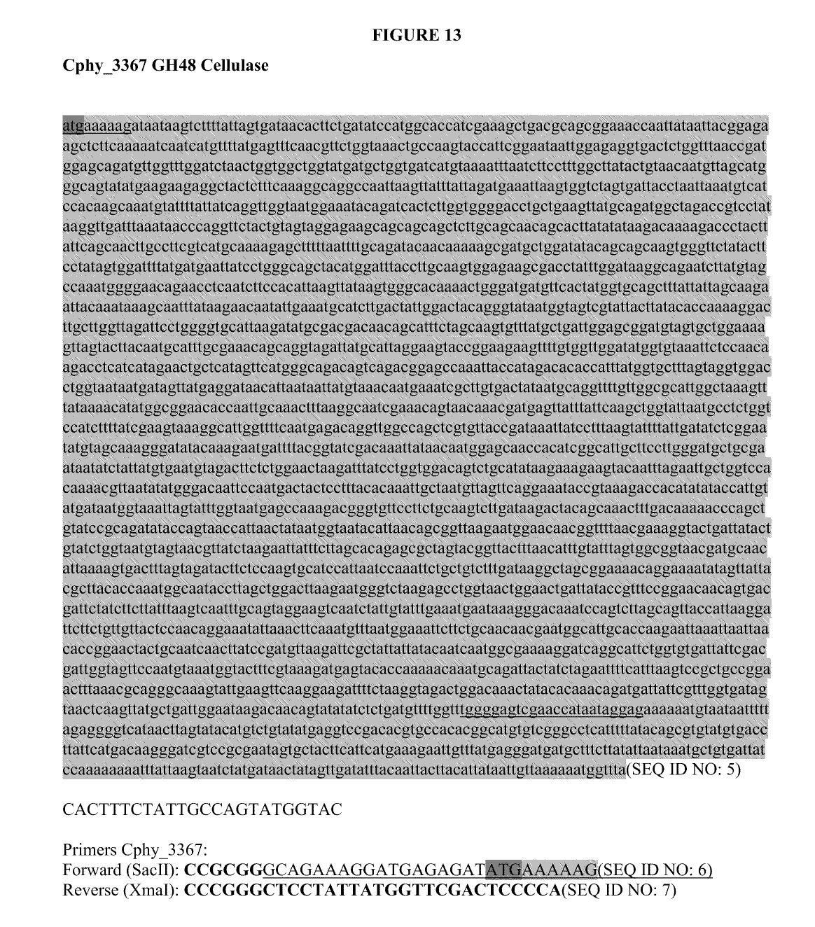

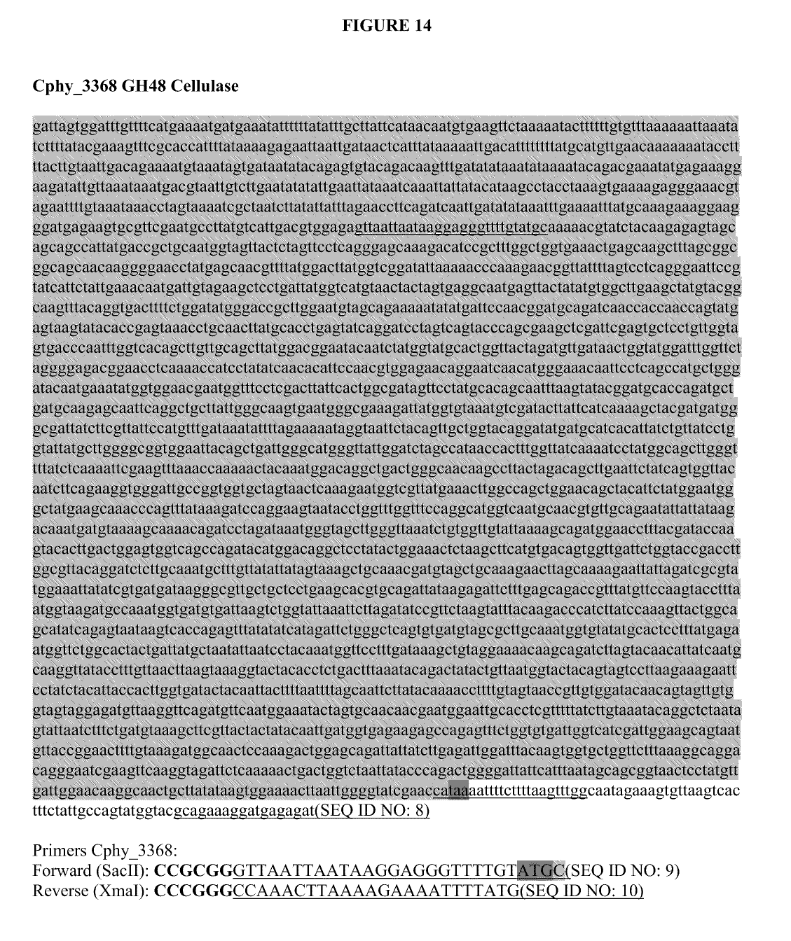

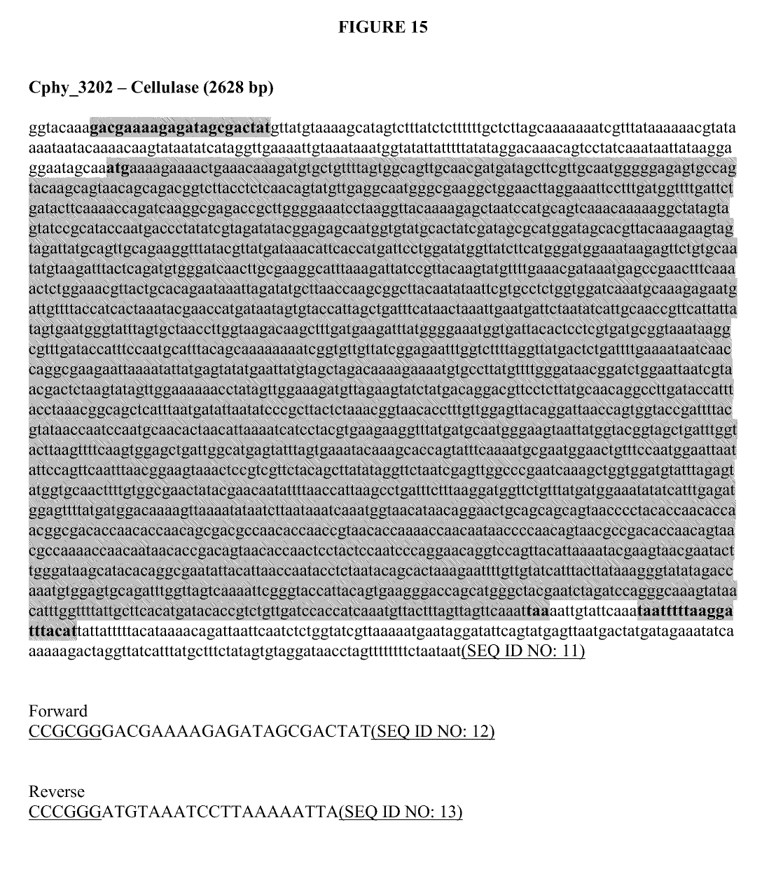

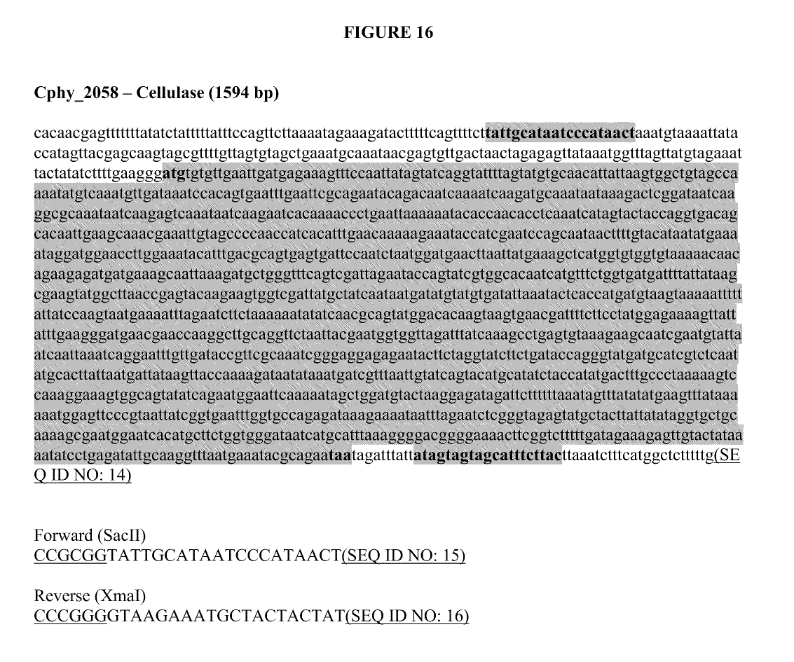

Using the methods above and the primers described in FIGS. 13, 14, 15, and 16, respectively, genes encoding Cphy_{—}3367, Cphy_{—}3368, Cphy_{—}3202 and Cphy_{—}2058 were cloned into pCphy3510 to produce pCphy3510_{—}3367, pCphy3510_{—}3368, pCphy3510_{—}3202, and pCphy3510_{—}2058 respectively. These vectors were transformed into C. phytofermentans via electroporation as described supra. In addition, genes encoding the heat shock chaperonin proteins, Cphy_{—}3289 (GroES, FIG. 15) and Cphy_{—}3290 (GroEL, FIG. 15) were incorporated into pCphy3510. In another embodiment, an endogenous or exogenous gene can be cloned into this vector and used to transform C. phytofermentans, another bacteria or fungal cell.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A process for producing a fermentive end-product comprising:
(a) contacting a carbonaceous biomass with:
i. a microorganism that hydrolyses and ferments said biomass; and
ii. an external source of one or more enzymes that are capable of enhancing said hydrolysis, and
(b) allowing sufficient time for said hydrolysis and fermentation to produce a fermentive end-product;
wherein said one or more enzymes do not include a xylanase, a hemicellulase, a glucanase or glucosidase, and wherein said external source is not said microorganism.

2. A process for producing a fermentive end-product comprising:
(a) contacting a carbonaceous biomass with:
i. a microorganism that hydrolyses and ferments said biomass; and
ii. an external source of one or more enzymes that are capable of enhancing said hydrolysis, and
(b) allowing sufficient time for said hydrolysis and fermentation to produce a fermentive end-product;
wherein said one or more enzymes comprise a cellulase, and wherein the cellulase and the microorganism act synergistically to enhance hydrolysis.

3. A process for producing a fermentive end-product comprising:
(a) contacting a carbonaceous biomass with:
i. a recombinant microorganism that hydrolyses and ferments said biomass; and
ii. an external source of one or more enzymes that are capable of enhancing said hydrolysis, and
(b) allowing sufficient time for said hydrolysis and fermentation to produce a fermentive end-product.

4. The process of claim 1, wherein said contact is in a large-scale fermentation vessel, wherein the fermentation vessel is adapted to provide suitable conditions for fermentation of one or more carbohydrate into fermentive end-products.

5. The process of claim 1, wherein said microorganism is capable of direct fermentation of C5 and C6 carbohydrates.

6. The process of claim 1, wherein said microorganism is a bacterium.

7. The process of claim 1, wherein said microorganism is a species of Clostridia.

8. The process of claim 1, wherein said microorganism is Clostridium phytofermentans.

9. The process of claim 1, wherein the microorganism is non-recombinant.

10. The process of claim 3, wherein the recombinant microorganism comprises one or more heterologous polynucleotides or one or more copies of an endogenous polynucleotide that enhance said activity of one or more cellulases.

11. The process of claim 1, wherein said biomass comprises one or more of corn steep solids, corn steep liquor, malt syrup, xylan, cellulose, hemicellulose, fructose, glucose, mannose, rhamnose, xylose, municipal waste, wood, plant material, plant material extract, a natural or synthetic polymer, switchgrass, bagasse, corn stover poplar or a combination thereof.

12. (canceled)

13. (canceled)

14. (canceled)

15. The process of claim 1, wherein said fermentive end-product is alcohol, ethanol, lactic acid, acetic acid or formic acid.

16. (canceled)

17. The process of claim 1, wherein said biomass comprises a higher concentration of oligomeric carbohydrates relative to monomeric carbohydrates.

18. The process of claim 10, wherein said one or more heterologous polynucleotides or said one or more copies of an endogenous polynucleotide comprises SEQ ID NO: 1, SEQ ID NO: 2, SEQ ID NO: 5, SEQ ID NO: 8, SEQ ID NO: 11, or SEQ ID NO: 14.

19. The process of claim 10, said one or more heterologous polynucleotides or said one or more copies of an endogenous polynucleotide encodes one or more enzymes selected from the group consisting of Cphy_{—}3202, Cphy_{—}2058, Cphy_{—}1163, Cphy_{—}3367, Cphy_{—}1100, Cphy_{—}1510, Cphy_{—}3368, and Cphy_{—}2128.

20. (canceled)

21. The process of claim 2, wherein said cellulase is in an amount from about 0.4 to about 15 filter paper unit (FPU)/gram cellulose.

22. The process of claim 1, wherein said enhanced activity is equivalent to addition of said cellulases in an amount sufficient to provide activity of about 0.4 to about 15 filter paper unit (FPU)/gram cellulose.

23. The process of claim 1, wherein said hydrolysis provides results in a greater concentration of cellobiose and/or larger oligomers, relative to monomeric carbohydrates.

24. (canceled)

25. The process of claim 1, wherein said biomass is pre-conditioned with alkali treatment.

26. The process of claim 3, wherein said recombinant microorganism produces one or more hydrolytic enzyme encoded by a variant having a polynucleotide sequence with an identity of 70% or more compared to a sequence selected from SEQ ID NO: 1, SEQ ID NO: 2, SEQ ID NO: 5, SEQ ID NO: 8, SEQ ID NO: 11, or SEQ ID NO: 14.


SEQUENCE LISTING (32 sequences)

SEQ ID NO: 1 - DNA, 4904 bp, Artificial Sequence
  Description of Artificial Sequence Synthetic polynucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaagctttggctaacacacacgccattccaaccaatagttttctcggcataaagccatgctctgacgcttaaatgcactaatgccttaaaaaaacattaaagtctaacacactagacttatttacttcgtaattaagtcgttaaaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctccttatattttatttcgatttatttgttatttatttaacatttttctattgacctcatcttttctatgtgttattcttttgttaattgtttacaaataatctacgatacatagaaggaggaaaaactagtatactagtatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgatccggccagcctcgcagagcaggattcccgttgagcaccgccaggtgcgaataagggacagtgaagaaggaacacccgctcgcgggtgggcctacttcacctatcctgcccggatcgattatgtcttttgcgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagcttagcttggctgcaggtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgccgagcgcagcgagtcagtgagcgaggaagcggaaga

SEQ ID NO: 2 - DNA, 1563 bp, Clostridium phytofermentans
ttttctagagatcactcatatagggagctataaaaaatattagaaaaaggggaaatttaattatgagtaaaatcagaagaatagtttcattgtttttgattgtaacaatctttatcacgacatgcttttttaatgttggtcaaaaggtatatgctgctgatacgaataatgatgattggctacattgtgtaggcaataaaatttatgacatgaatggcaatgaggtttggctgaccggtgcgaattggtttggttttaactgtactgaaaatgtatttcatggtgcatggtacgatattaaggggatgttaactaatattgcaaacagaggaataggatttttaagagttccaatttcaacggaacttttgtatagttggatgataggcaaacctaataaagtttcaagtgtgaccgctgtcaataatccaccttattatgtatgcaaccctgatttttatgatcctacaacaaatagtgttaaaaatagtatggaaatatttgatatcattatgggatactgcaaacaattggggatcaaagtaatggtagatgttcatagtccggatgcaaataattcaggtcataactatccgttatggtatgggttaactacgactactgcaggtgaaataacgacagataagtggatcaatactcaagcatggctggctgataaatacaaaaatgacgatactattctggcatttgatataaaaaatgaacctcatggacagaggggatatagtactacaacacctactaatatagcaaaatgggataattccacagatgagaataactggaagtatgcggcggaaagatgtgcgaaagctatacttgctaaaaaccctaaattattaattatgattgaaggtgttgaacaataccctaaaactgaaaaaggttataactataatacaccggatgtatggggagctactggtgatcagtctccatggtatagtgcttggtggggtggaaatttaagaggagtaaaggattatccaattaatataggcactctcaatagtcagatcgtctattcccctcatgactatggtccttccgtatacaaccaaccatggtttgataaggattttacaactcagaccctattagatgattattggtataatacttgggcatatattaaagataaaggtattgcaccacttttgataggtgagtggggaggttttatggatggtggaaagaaccagaaatggatgacattattaagagattatatagtaaataatcgtatccaccatacattctggtgtatcaatccgaactcaggggatactggaggtttactaggatatgattggcaaacttgggatgaagcaaaatacgctttattaaaacctgcattatggcagtcaaatggtaaatttattggtctagaccatcagacacctcttggtgtaaatggtatatcattagggcaatattatggaaaataaatgaacaaaaaaataaaataatttatagagaatgggttcaaatcgagcccattcttttttgtgcgaagccgtagccttaagaaacttttttggt

SEQ ID NO: 3 - DNA, 45 bp, Artificial Sequence
  Description of Artificial Sequence Synthetic primer
ccgcggaggagggttttgtatgagtaaaatcagaagaatagtttc

SEQ ID NO: 4 - DNA, 51 bp, Artificial Sequence
  Description of Artificial Sequence Synthetic primer
cccgggttagtggtggtggtggtggtgttttccataatattgccctaatga

SEQ ID NO: 5 - DNA, 3267 bp, Clostridium phytofermentans
atgaaaaagataataagtcttttattagtgataacacttctgatatccatggcaccatcgaaagctgacgcagcggaaaccaattataattacggagaagctcttcaaaaatcaatcatgttttatgagtttcaacgttctggtaaactgccaagtaccattcggaataattggagaggtgactctggtttaaccgatggagcagatgttggtttggatctaactggtggctggtatgatgctggtgatcatgtaaaatttaatcttcctttggcttatactgtaacaatgttagcatgggcagtatatgaagaagaggctactctttcaaaggcaggccaattaagttatttattagatgaaattaagtggtctagtgattacctaattaaatgtcatccacaagcaaatgtattttattatcaggttggtaatggaaatacagatcactcttggtggggacctgctgaagttatgcagatggctagaccgtcctataaggttgatttaaataacccaggttctactgtagtaggagaagcagcagcagctcttgcagcaacagcacttatatataagacaaaagaccctacttattcagcaacttgccttcgtcatgcaaaagagctttttaattttgcagatacaacaaaaagcgatgctggatatacagcagcaagtgggttctatacttcctatagtggattttatgatgaattatcctgggcagctacatggatttaccttgcaagtggagaagcgacctatttggataaggcagaatcttatgtagccaaatggggaacagaacctcaatcttccacattaagttataagtgggcacaaaactgggatgatgttcactatggtgcagctttattattagcaagaattacaaataaagcaatttataagaacaatattgaaatgcatcttgactattggactacagggtataatggtagtcgtattacttatacaccaaaaggacttgcttggttagattcctggggtgcattaagatatgcgacgacaacagcatttctagcaagtgtttatgctgattggagcggatgtagtgctggaaaagttagtacttacaatgcatttgcgaaacagcaggtagattatgcattaggaagtaccggaagaagttttgtggttggatatggtgtaaattctccaacaagacctcatcatagaactgctcatagttcatgggcagacagtcagacggagccaaattaccatagacacaccatttatggtgctttagtaggtggacctggtaataatgatagttatgaggataacattaataattatgtaaacaatgaaatcgcttgtgactataatgcaggttttgttggcgcattggctaaagtttataaaacatatggcggaacaccaattgcaaactttaaggcaatcgaaacagtaacaaacgatgagttatttattcaagctggtattaatgcctctggtccatcttttatcgaagtaaaggcattggttttcaatgagacaggttggccagctcgtgttaccgataaattatcctttaagtattttattgatatctcggaatatgtagcaaagggatatacaaagaatgattttacggtatcgacaaattataacaatggagcaaccacatcggcattgcttccttgggatgctgcgaataatatctattatgtgaatgtagacttctctggaactaagatttatcctggtggacagtctgcatataagaaagaagtacaatttagaattgctggtccacaaaacgttaatatatgggacaattccaatgactactcctttacacaaattgctaatgttagttcaggaaataccgtaaagaccacatatataccattgtatgataatggtaaattagtatttggtaatgagccaaagacgggtgttccttctgcaagtcttgataagactacagcaaactttgacaaaaacccagctgtatccgcagatataccagtaaccattaactataatggtaatacattaacagcggttaagaatggaacaacggttttaacgaaaggtactgattatactgtatctggtaatgtagtaacgttatctaagaattatttcttagcacagagcgctagtacggttactttaacatttgtatttagtggcggtaacgatgcaacattaaaagtgactttagtagatacttctccaagtgcatccattaatccaaattctgctgtctttgataaggctagcggaaaacaggaaaatatagttattacgcttacaccaaatggcaataccttagctggacttaagaatgggtctaagagcctggtaactggaactgattataccgtttccggaacaacagtgacgattctatcttcttatttaagtcaatttgcagtaggaagtcaatctattgtatttgaaatgaataaagggacaaatccagtcttagcagttaccattaaggattcttctgttgttactccaacaggaaatattaaacttcaaatgtttaatggaaattcttctgcaacaacgaatggcattgcaccaagaattaaattaattaacaccggaactactgcaatcaacttatccgatgttaagattcgctattattatacaatcaatggcgaaaaggatcaggcattctggtgtgattattcgacgattggtagttccaatgtaaatggtactttcgtaaagatgagtacaccaaaaacaaatgcagattactatctagaattttcatttaagtccgctgccggaactttaaacgcagggcaaagtattgaagttcaaggaagattttctaaggtagactggacaaactatacacaaacagatgattattcgtttggtgatagtaactcaagttatgctgattggaataagacaacagtatatatctctgatgttttggtttggggagtcgaaccataataggagaaaaaatgtaataatttttagaggggtcataacttagtatacatgtctgtatatgaggtccgacacgtgccacacggcatgtgtcgggcctcatttttatacagcgtgtatgtgaccttattcatgacaagggatcgtccgcgaatagtgctacttcattcatgaaagaattgtttatgagggatgatgctttcttatattaataaatgctgtgattatccaaaaaaaatttattaagtaatctatgataactatagttgatatttacaattacttacattataattgttaaaaaatggttta

SEQ ID NO: 6 - DNA, 33 bp, Artificial Sequence
  Description of Artificial Sequence Synthetic primer
ccgcgggcagaaaggatgagagatatgaaaaag

SEQ ID NO: 7 - DNA, 30 bp, Artificial Sequence
  Description of Artificial Sequence Synthetic primer
cccgggctcctattatggttcgactcccca

SEQ ID NO: 8 - DNA, 3421 bp, Clostridium phytofermentans
gattagtggatttgttttcatgaaaatgatgaaatattttttatatttgcttattcataacaatgtgaagttctaaaaatacttttttgtgtttaaaaaattaaatatcttttatacgaaagtttcgcaccattttataaaagagaattaattgataactcatttataaaaattgacattttttttatgcatgttgaacaaaaaaataccttttacttgtaattgacagaaaatgtaaatagtgataatatacagagtgtacagacaagtttgatatataaatataaaatacagacgaaatatgagaaaggaagatattgttaaataaatgacgtaattgtcttgaatatatattgaattataaatcaaattattatacataagcctacctaaagtgaaaagagggaaacgtagaattttgtaaataaacctagtaaaatcgctaatcttatattatttagaaccttcagatcaattgatatataaatttgaaaatttatgcaaagaaaggaagggatgagaagtgcgttcgaatgccttatgtcattgacgtggagagttaattaataaggagggttttgtatgcaaaaacgtatctacaagagagtagcagcagccattatgaccgctgcaatggtagttactctagttcctcagggagcaaagacatccgctttggctggtgaaactgagcaagctttagcggcggcagcaacaaggggaacctatgagcaacgttttatggacttatggtcggatattaaaaacccaaagaacggttattttagtcctcagggaattccgtatcattctattgaaacaatgattgtagaagctcctgattatggtcatgtaactactagtgaggcaatgagttactatatgtggcttgaagctatgtacggcaagtttacaggtgacttttctggatatgggaccgcttggaatgtagcagaaaaatatatgattccaacggatgcagatcaaccaccaaccagtatgagtaagtatacaccgagtaaacctgcaacttatgcacctgagtatcaggatcctagtcagtacccagcgaagctcgattcgagtgctcctgttggtagtgacccaatttggtcacagcttgttgcagcttatggacggaatacaatctatggtatgcactggttactagatgttgataactggtatggatttggttctaggggagacggaacctcaaaaccatcctatatcaacacattccaacgtggagaacaggaatcaacatgggaaacaattcctcagccatgctgggatacaatgaaatatggtggaacgaatggtttcctcgacttattcactggcgatagttcctatgcacagcaatttaagtatacggatgcaccagatgctgatgcaagagcaattcaggctgcttattgggcaagtgaatgggcgaaagattatggtgtaaatgtcgatacttattcatcaaaagctacgatgatgggcgattatcttcgttattccatgtttgataaatattttagaaaaataggtaattctacagttgctggtacaggatatgatgcatcacattatctgttatcctggtattatgcttggggcggtggaattacagctgattgggcatgggttattggatctagccataaccactttggttatcaaaatcctatggcagcttgggttttatctcaaaattcgaagtttaaaccaaaaactacaaatggacaggctgactgggcaacaagccttactagacagcttgaattctatcagtggttacaatcttcagaaggtgggattgccggtggtgctagtaactcaaagaatggtcgttatgaaacttggccagctggaacagctacattctatggaatgggctatgaagcaaacccagtttataaagatccaggaagtaatacctggtttggtttccaggcatggtcaatgcaacgtgttgcagaatattattataagacaaatgatgtaaaagcaaaacagatcctagataaatgggtagcttgggttaaatctgtggttgtattaaaagcagatggaacctttacgataccaagtacacttgactggagtggtcagccagatacatggacaggctcctatactggaaactctaagcttcatgtgacagtggttgattctggtaccgaccttggcgttacaggatctcttgcaaatgctttgttatattatagtaaagctgcaaacgatgtagctgcaaagaacttagcaaaagaattattagatcgcgtatggaaattatatcgtgatgataagggcgttgctgctcctgaagcacgtgcagattataagagattctttgagcagaccgtttatgttccaagtacctttaatggtaagatgccaaatggtgatgtgattaagtctggtattaaattcttagatatccgttctaagtatttacaagacccatcttatccaaagttactggcagcatatcagagtaataagtcaccagagtttatatatcatagattctgggctcagtgtgatgtagcgcttgcaaatggtgtatatgcactcctttatgagaatggttctggcactactgattatgctaatattaatcctacaaatggttcctttgataaagctgtaggaaaacaagcagatcttagtacaacattatcaatgcaaggttatacctttgttaacttaagtaaaggtactacacctctgactttaaatacagactatactgttaatggtactacagtagtccttaagaaagaattcctatctacattaccacttggtgatactacaattacttttaattttagcaattcttatacaaaaccttttgtagtaaccgttgtggatacaacagtagttgtggtagtaggagatgttaaggttcagatgttcaatggaaatactagtgcaacaacgaatggaattgcacctcgtttttatcttgtaaatacaggctctaatagtattaatctttctgatgtaaagcttcgttactactatacaattgatggtgagaagagccagagtttctggtgtgattggtcatcgattggaagcagtaatgttaccggaacttttgtaaagatggcaactccaaagactggagcagattattatcttgagattggatttacaagtggtgctggttctttaaaggcaggacagggaatcgaagttcaaggtagattctcaaaaactgactggtctaattatacccagactggggattattcatttaatagcagcggtaactcctatgttgattggaacaaggcaactgcttatataagtggaaaacttaattggggtatcgaaccataaaattttcttttaagtttggcaatagaaagtgttaagtcactttctattgccagtatggtacgcagaaaggatgagagat

SEQ ID NO: 9 - DNA, 34 bp, Artificial Sequence
  Description of Artificial Sequence Synthetic primer
ccgcgggttaattaataaggagggttttgtatgc

SEQ ID NO: 10 - DNA, 30 bp, Artificial Sequence
  Description of Artificial Sequence Synthetic primer
cccgggccaaacttaaaagaaaattttatg

SEQ ID NO: 11 - DNA, 2636 bp, Clostridium phytofermentans
ggtacaaagacgaaaagagatagcgactatgttatgtaaaagcatagtctttatctcttttttgctcttagcaaaaaaatcgtttataaaaaacgtataaaataatacaaaacaagtataatatcataggttgaaaattgtaaataaatggtatattatttttatataggacaaacagtcctatcaaataattataaggaggaatagcaaatgaaaagaaaactgaaacaaagatgtgctgttttagtggcagttgcaacgatgatagcttcgttgcaatgggggagagtgccagtacaagcagtaacagcagacggtcttacctctcaacagtatgttgaggcaatgggcgaaggctggaacttaggaaattcctttgatggttttgattctgatacttcaaaaccagatcaaggcgagaccgcttggggaaatcctaaggttacaaaagagctaatccatgcagtcaaacaaaaaggctatagtagtatccgcataccaatgaccctatatcgtagatatacggagagcaatggtgtatgcactatcgatagcgcatggatagcacgttacaaagaagtagtagattatgcagttgcagaaggtttatacgttatgataaacattcaccatgattcctggatatggttatcttcatgggatggaaataagagttctgtgcaatatgtaagatttactcagatgtgggatcaacttgcgaaggcatttaaagattatccgttacaagtatgttttgaaacgataaatgagccgaactttcaaaactctggaaacgttactgcacagaataaattagatatgcttaaccaagcggcttacaatataattcgtgcctctggtggatcaaatgcaaagagaatgattgttttaccatcactaaatacgaaccatgataatagtgtaccattagctgatttcataactaaattgaatgattctaatatcattgcaaccgttcattattatagtgaatgggtatttagtgctaaccttggtaagacaagctttgatgaagatttatggggaaatggtgattacactcctcgtgatgcggtaaataaggcgtttgataccatttccaatgcatttacagcaaaaaaaatcggtgttgttatcggagaatttggtcttttaggttatgactctgattttgaaaataatcaaccaggcgaagaattaaaatattatgagtatatgaattatgtagctagacaaaagaaaatgtgccttatgttttgggataacggatctggaattaatcgtaacgactctaagtatagttggaaaaaacctatagttggaaagatgttagaagtatctatgacaggacgttcctcttatgcaacaggccttgataccatttacctaaacggcagctcatttaatgatattaatatcccgcttactctaaacggtaacacctttgttggagttacaggattaaccagtggtaccgattttacgtataaccaatccaatgcaacactaacattaaaatcatcctacgtgaagaaggtttatgatgcaatgggaagtaattatggtacggtagctgatttggtacttaagttttcaagtggagctgattggcatgagtatttagtgaaatacaaagcaccagtatttcaaaatgcgaatggaactgtttccaatggaattaatattccagttcaatttaacggaagtaaactccgtcgttctacagcttatataggttctaatcgagttggcccgaatcaaagctggtggatgtatttagagtatggtgcaacttttgtggcgaactatacgaacaatattttaaccattaagcctgatttctttaaggatggttctgtttatgatggaaatatatcatttgagatggagttttatgatggacaaaagttaaaatataatcttaataaatcaaatggtaacataacaggaactgcagcagcagtaacccctacaccaacaccaacggcgacaccaacaccaacagcgacgccaacaccaaccgtaacaccaaaaccaacaataaccccaacagtaacgccgacaccaacagtaacgccaaaaccaacaataacaccgacagtaacaccaactcctactccaatcccaggaacaggtccagttacattaaaatacgaagtaacgaatacttgggataagcatacacaggcgaatattacattaaccaatacctctaatacagcactaaagaattttgttgtatcatttacttataaagggtatatagaccaaatgtggagtgcagatttggttagtcaaaattcgggtaccattacagtgaagggaccagcatgggctacgaatctagatccagggcaaagtataacatttggttttattgcttcacatgatacaccgtctgttgatccaccatcaaatgttactttagttagttcaaattaaaattgtattcaaataatttttaaggatttacattattatttttacataaaacagattaattcaatctctggtatcgttaaaaatgaataggatattcagtatgagttaatgactatgatagaaatatcaaaaaagactaggttatcatttatgctttctatagtgtaggataacctagttttttttctaataat

SEQ ID NO: 12 - DNA, 28 bp, Artificial Sequence
  Description of Artificial Sequence Synthetic primer
ccgcgggacgaaaagagatagcgactat

SEQ ID NO: 13 - DNA, 26 bp, Artificial Sequence
  Description of Artificial Sequence Synthetic primer
cccgggatgtaaatccttaaaaatta

SEQ ID NO: 14 - DNA, 1688 bp, Clostridium phytofermentans
cacaacgagtttttttatatctatttttatttccagttcttaaaatagaaagatactttttcagttttcttattgcataatcccataactaaatgtaaaattataccatagttacgagcaagtagcgttttgttagtgtagctgaaatgcaaataacgagtgttgactaactagagagttataaatggtttagttatgtagaaattactatatcttttgaagggatgtgtgttgaattgatgagaaagtttccaattatagtatcaggtattttagtatgtgcaacattattaagtggctgtagccaaaatatgtcaaatgttgataaatccacagtgaatttgaattcgcagaatacagacaatcaaaatcaagatgcaaataataaagactcggataatcaaggcgcaaataatcaagagtcaaataatcaagaatcacaaaaccctgaattaaaaaatacaccaacacctcaaatcatagtactaccaggtgacagcacaattgaagcaaacgaaattgtagccccaaccatcacatttgaacaaaaagaaataccatcgaatccagcaataacttttgtacataatatgaaaataggatggaaccttggaaatacatttgacgcagtgagtgattccaatctaatggatgaacttaattatgaaagctcatggtgtggtgtaaaaacaacagaagagatgatgaaagcaattaaagatgctgggtttcagtcgattagaataccagtatcgtggcacaatcatgtttctggtgatgattttattataagcgaagtatggcttaaccgagtacaagaagtggtcgattatgctatcaataatgatatgtatgtgatattaaatactcaccatgatgtaagtaaaaatttttattatccaagtaatgaaaatttagaatcttctaaaaaatatatcaacgcagtatggacacaagtaagtgaacgattttcttcctatggagaaaagttattatttgaagggatgaacgaaccaaggcttgcaggttctaattacgaatggtggttagatttatcaaagcctgagtgtaaagaagcaatcgaatgtattaatcaattaaatcaggaatttgttgataccgttcgcaaatcgggaggagagaatacttctaggtatcttctgataccagggtatgatgcatcgtctcaatatgcacttattaatgattataagttaccaaaagataatataaatgatcgtttaattgtatcagtacatgcatatctaccatatgactttgccctaaaaagtccaaaggaaagtggcagtatatcagaatggaattcaaaaatagctggatgtactaaggagatagattcttttttaaatagtttatatatgaagtttataaaaaatggagttcccgtaattatcggtgaatttggtgccagagataaagaaaataatttagaatctcgggtagagtatgctacttattatataggtgctgcaaaagcgaatggaatcacatgcttctggtgggataatcatgcatttaaaggggacggggaaaacttcggtctttttgatagaaagagttgtactataaaatatcctgagatattgcaaggtttaatgaaatacgcagaataatagatttattatagtagtagcatttcttacttaaatctttcatggctctttttg

SEQ ID NO: 15 - DNA, 26 bp, Artificial Sequence
  Description of Artificial Sequence Synthetic primer
ccgcggtattgcataatcccataact

SEQ ID NO: 16 - DNA, 26 bp, Artificial Sequence
  Description of Artificial Sequence Synthetic primer
cccggggtaagaaatgctactactat

SEQ ID NO: 17 - DNA, 2330 bp, Clostridium phytofermentans
attgaagtaaaatttactaatatatactcaacgattagcactcgaatagagtgagtgctaataactgatatgatacagtattaaaggaggaaataaatatgaaattagtgccattaggagacaaagttgtattaaagcaactcgtagcagaagagactacaaagtcgggtattgtattacctggtcaggcaaaagaaaaaccacaacaagcagaggttgttgcagttggccctggtggaatggtggatggaaaagaagtaaccatgcaggttaaagttggcgacaaagttatttattccaaatatgctggaactgaagtaaaattagatgaagaagaatttatcgttgtaaaacaaaacgatatcgtagcaattgtagcagattaataataataaagtgtgaatcatatttttatgattcatataaataaaacgtgaaaatataatcagaattattataaattaaaacttggaggaatacaattatggctaaggatattaaatatagtgcagatgccagagtagcgatggaagctggtgtaaacaagttagcaaatacagtaagagtaactctaggaccaaagggaagaaatgtagttcttgataagtccttcggtgcaccattaattacaaatgatggtgttactattgcaaaagaaattgagttagaagattcctttgagaatatgggcgctcagcttgtaaaagaagttgctacaaagacaaatgatgtagcaggtgatggtacaacaacagctactgttcttgctcaagctatgattaacgaaggtatgaaaaatcttgcagcaggtgctaatccgatcatcttaagaagaggtatgaagaaagctactgattgcgctgttgaagcaattagcagtatgagctcagcaattaatggtaaagatcagattgctaaggttgctgcaatctctgctggcgatgattccgtaggtgagatggttgcggatgctatggataaagttagcaaagatggtgttatcaccattgaagagtctaagactatgcagactgagcttgacttagtagaaggtatgcaatttgaccgtggatatgtttccgcatatatggcgactgatatggataagatggaagcaaatctagataatccatatattttaatcacagataagaagatcagcaacattcaggagatccttcctgttcttgagcagattgttcaaagtggatccagattattaatcatcgctgaagatatcgaaggcgaagctttaacaacattagtaatcaataagttaagagggacattcactgttgttggtgttaaggcgccaggttatggtgatagaagaaaggctatgttacaagatatcgctattttaactggtggtactgttatctctgatgaacttggccttgacttaaaagaagctacattagatcagcttggtcgtgcaaaatccgttaaaattcagaaagaaaacactatcattgttgatggtgaaggaaataaagcagaaatcgaagctagaatttctcagattaaggctcagattgctgaaacaacatcagaatttgataaagaaaaattacaggagagacttgctaaacttgcaggtggtgtagctgtaattcgtgttggtgctgcaacagagactgagatgaaagagaagaagcttcgtatggaagatgctttagcagctacaagagcagctgtggaagaaggtattatcgcaggtggcggttctgcttacatccatgcatctaaggaagttgctaaacttgctgctaaattagaaggtgatgagagaactggtgcacagattatattaaaagcattagaagctccattatcatgcatcgctcaaaacgctggtttagaaggcgctgttattgttaacaaggttagagaaaagaaaacaggtgttggtttcaatgccctaactgagaagtatgtagatatggtagaagacggaattcttgatccttctaaggttacaagaagtgctcttcagaatgcaaccagtgttgcttctacattcttaacaacagaagctgcagttgcatccattaaagaaccagctccagctatgccagcaggcggccctggcggaatgggtatgatgtaatttactgatataaaattaatatatcttaagactttagaatccatgtataagtatgagtatacatggattctttcagcgtataggaaatattgttgtaattacctataaacaagaaaaagtacgttttcgatatgaagtatattcgcattattccttaaaagataataaagtccttttacttttataaataatttagtataataaaggtagcaaggaatcg

SEQ ID NO: 18 - DNA, 31 bp, Artificial Sequence
  Description of Artificial Sequence Synthetic primer
ccgcggttaaaggaggaaataaatatgaaat

SEQ ID NO: 19 - DNA, 32 bp, Artificial Sequence
  Description of Artificial Sequence Synthetic primer
cccgggattaattttatatcagtaaattacat

SEQ ID NO: 20 - protein, 468 aa, Clostridium phytofermentans
MSKIRRIVSLFLIVTIFITTCFFNVGQKVYAADTNNDDWLHCVGNKIYDMNGNEVWLTGANWFGFNCTENVFHGAWYDIKGMLTNIANRGIGFLRVPISTELLYSWMIGKPNKVSSVTAVNNPPYYVCNPDFYDPTTNSVKNSMEIFDIIMGYCKQLGIKVMVDVHSPDANNSGHNYPLWYGLTTTTAGEITTDKWINTQAWLADKYKNDDTILAFDIKNEPHGQRGYSTTTPTNIAKWDNSTDENNWKYAAERCAKAILAKNPKLLIMIEGVEQYPKTEKGYNYNTPDVWGATGDQSPWYSAWWGGNLRGVKDYPINIGTLNSQIVYSPHDYGPSVYNQPWFDKDFTTQTLLDDYWYNTWAYIKDKGIAPLLIGEWGGFMDGGKNQKWMTLLRDYIVNNRIHHTFWCINPNSGDTGGLLGYDWQTWDEAKYALLKPALWQSNGKFIGLDHQTPLGVNGISLGQYYGK

SEQ ID NO: 21 - protein, 985 aa, Clostridium phytofermentans
MKKIISLLLVITLLISMAPSKADAAETNYNYGEALQKSIMFYEFQRSGKLPSTIRNNWRGDSGLTDGADVGLDLTGGWYDAGDHVKFNLPLAYTVTMLAWAVYEEEATLSKAGQLSYLLDEIKWSSDYLIKCHPQANVFYYQVGNGNTDHSWWGPAEVMQMARPSYKVDLNNPGSTVVGEAAAALAATALIYKTKDPTYSATCLRHAKELFNFADTTKSDAGYTAASGFYTSYSGFYDELSWAATWIYLASGEATYLDKAESYVAKWGTEPQSSTLSYKWAQNWDDVHYGAALLLARITNKAIYKNNIEMHLDYWTTGYNGSRITYTPKGLAWLDSWGALRYATTTAFLASVYADWSGCSAGKVSTYNAFAKQQVDYALGSTGRSFVVGYGVNSPTRPHHRTAHSSWADSQTEPNYHRHTIYGALVGGPGNNDSYEDNINNYVNNEIACDYNAGFVGALAKVYKTYGGTPIANFKAIETVTNDELFIQAGINASGPSFIEVKALVFNETGWPARVTDKLSFKYFIDISEYVAKGYTKNDFTVSTNYNNGATTSALLPWDAANNIYYVNVDFSGTKIYPGGQSAYKKEVQFRIAGPQNVNIWDNSNDYSFTQIANVSSGNTVKTTYIPLYDNGKLVFGNEPKTGVPSASLDKTTANFDKNPAVSADIPVTINYNGNTLTAVKNGTTVLTKGTDYTVSGNVVTLSKNYFLAQSASTVTLTFVFSGGNDATLKVTLVDTSPSASINPNSAVFDKASGKQENIVITLTPNGNTLAGLKNGSKSLVTGTDYTVSGTTVTILSSYLSQFAVGSQSIVFEMNKGTNPVLAVTIKDSSVVTPTGNIKLQMFNGNSSATTNGIAPRIKLINTGTTAINLSDVKIRYYYTINGEKDQAFWCDYSTIGSSNVNGTFVKMSTPKTNADYYLEFSFKSAAGTLNAGQSIEVQGRFSKVDWTNYTQTDDYSFGDSNSSYADWNKTTVYISDVLVWGVEP

SEQ ID NO: 22 - protein, 919 aa, Clostridium phytofermentans
MQKRIYKRVAAAIMTAAMVVTLVPQGAKTSALAGETEQALAAAATRGTYEQRFMDLWSDIKNPKNGYFSPQGIPYHSIETMIVEAPDYGHVTTSEAMSYYMWLEAMYGKFTGDFSGYGTAWNVAEKYMIPTDADQPPTSMSKYTPSKPATYAPEYQDPSQYPAKLDSSAPVGSDPIWSQLVAAYGRNTIYGMHWLLDVDNWYGFGSRGDGTSKPSYINTFQRGEQESTWETIPQPCWDTMKYGGTNGFLDLFTGDSSYAQQFKYTDAPDADARAIQAAYWASEWAKDYGVNVDTYSSKATMMGDYLRYSMFDKYFRKIGNSTVAGTGYDASHYLLSWYYAWGGGITADWAWVIGSSHNHFGYQNPMAAWVLSQNSKFKPKTTNGQADWATSLTRQLEFYQWLQSSEGGIAGGASNSKNGRYETWPAGTATFYGMGYEANPVYKDPGSNTWFGFQAWSMQRVAEYYYKTNDVKAKQILDKWVAWVKSVVVLKADGTFTIPSTLDWSGQPDTWTGSYTGNSKLHVTVVDSGTDLGVTGSLANALLYYSKAANDVAAKNLAKELLDRVWKLYRDDKGVAAPEARADYKRFFEQTVYVPSTFNGKMPNGDVIKSGIKFLDIRSKYLQDPSYPKLLAAYQSNKSPEFIYHRFWAQCDVALANGVYALLYENGSGTTDYANINPTNGSFDKAVGKQADLSTTLSMQGYTFVNLSKGTTPLTLNTDYTVNGTTVVLKKEFLSTLPLGDTTITFNFSNSYTKPFVVTVVDTTVVVVVGDVKVQMFNGNTSATTNGIAPRFYLVNTGSNSINLSDVKLRYYYTIDGEKSQSFWCDWSSIGSSNVTGTFVKMATPKTGADYYLEIGFTSGAGSLKAGQGIEVQGRFSKTDWSNYTQTGDYSFNSSGNSYVDWNKATAYISGKLNWGIEP

SEQ ID NO: 23 - protein, 743 aa, Clostridium phytofermentans
MKRKLKQRCAVLVAVATMIASLQWGRVPVQAVTADGLTSQQYVEAMGEGWNLGNSFDGFDSDTSKPDQGETAWGNPKVTKELIHAVKQKGYSSIRIPMTLYRRYTESNGVCTIDSAWIARYKEVVDYAVAEGLYVMINIHHDSWIWLSSWDGNKSSVQYVRFTQMWDQLAKAFKDYPLQVCFETINEPNFQNSGNVTAQNKLDMLNQAAYNIIRASGGSNAKRMIVLPSLNTNHDNSVPLADFITKLNDSNIIATVHYYSEWVFSANLGKTSFDEDLWGNGDYTPRDAVNKAFDTISNAFTAKKIGVVIGEFGLLGYDSDFENNQPGEELKYYEYMNYVARQKKMCLMFWDNGSGINRNDSKYSWKKPIVGKMLEVSMTGRSSYATGLDTIYLNGSSFNDINIPLTLNGNTFVGVTGLTSGTDFTYNQSNATLTLKSSYVKKVYDAMGSNYGTVADLVLKFSSGADWHEYLVKYKAPVFQNANGTVSNGINIPVQFNGSKLRRSTAYIGSNRVGPNQSWWMYLEYGATFVANYTNNILTIKPDFFKDGSVYDGNISFEMEFYDGQKLKYNLNKSNGNITGTAAAVTPTPTPTATPTPTATPTPTVTPKPTITPTVTPTPTVTPKPTITPTVTPTPTPIPGTGPVTLKYEVTNTWDKHTQANITLTNTSNTALKNFVVSFTYKGYIDQMWSADLVSQNSGTITVKGPAWATNLDPGQSITFGFIASHDTPSVDPPSNVTLVSSN

SEQ ID NO: 24 - protein, 469 aa, Clostridium phytofermentans
MCVELMRKFPIIVSGILVCATLLSGCSQNMSNVDKSTVNLNSQNTDNQNQDANNKDSDNQGANNQESNNQESQNPELKNTPTPQIIVLPGDSTIEANEIVAPTITFEQKEIPSNPAITFVHNMKIGWNLGNTFDAVSDSNLMDELNYESSWCGVKTTEEMMKAIKDAGFQSIRIPVSWHNHVSGDDFIISEVWLNRVQEVVDYAINNDMYVILNTHHDVSKNFYYPSNENLESSKKYINAVWTQVSERFSSYGEKLLFEGMNEPRLAGSNYEWWLDLSKPECKEAIECINQLNQEFVDTVRKSGGENTSRYLLIPGYDASSQYALINDYKLPKDNINDRLIVSVHAYLPYDFALKSPKESGSISEWNSKIAGCTKEIDSFLNSLYMKFIKNGVPVIIGEFGARDKENNLESRVEYATYYIGAAKANGITCFWWDNHAFKGDGENFGLFDRKSCTIKYPEILQGLMKYAE

SEQ ID NO: 25 - protein, 340 aa, Clostridium phytofermentans
METKEYIIKTLETLVNIPSPSGFTKEVMEFVEREANKYHYSCEYIKRGGLIITVPGESNDCLGLSAHVDTLGAMVRSIGGDGIIRFTCVGGYTMNSIEGEYCKIHTREGKVYTGTILSKSPSVHSYDDARTLERKDRNMEIRIDEKVENKEDVLNLGINNGDYISFDARFEYTKSGFIKSRHLDDKASVAVLLGLLKDLSEQNVTPKRTLKILISNLEEIGMGASYIPSEISEFIAVDMGAIGDDLAGNEYSVSICALDSSGPYDYELTTKLMNLAKDKQIPYVVDIFPHYGSDASAAMRGGNNIRAALIGQGIHASHGMERTHETALIATLDLLKVYVG

SEQ ID NO: 26 - protein, 521 aa, Clostridium phytofermentans
AMLQKMNGKVKKILGISIAFLMLIMVIPTSIAKAATNKTYDFNSMTYQSTWGVTYSISNGSGTFNFTGQYREIKFNLPETLDMSQCTSVTFNASSPNGQIAFKLYDTSGNQVAVVYNFNSNTSDCTFAPNSTAKVNSIGIMAQGTNNYSAVVNRVTFTMTGGSSGTGSSTLLNTYGNILKNSGTAVNLSQLQNSNTLSVIKTQYNSITLENEMKPDAVLGSSSTLMTVAQAKSNGYYIPSSYTESTVPTLKFSTIDAVLQICYNNGLKLRGHTLVWHSQTPDWFFRTGYSSSGSYVSQAVMDARMEMFIRSYMSHIYNGSYGSVVYAWDVVNEYLHASTSGWSQVYGSNLGTTPSYVKKAFQYAYDCLSSFGLTNSVKLFYNDYNTYEVTDQILSLVNFINSGTKLCAGVGMQSHLNTSYPSVSAYKTAMQKFLNAGYEVQVTELDVTNTSASTQATYVYDLMTAILSLKKAGGNITGITWWGLYDSVSWRASQNPLLFSNLTTPKESYNKALQAFTDAGY

SEQ ID NO: 27 - protein, 857 aa, Clostridium phytofermentans
MKGLVKKVSTFIIALSLLFSFITVPVSAGTALFVIESENTRLSSELQVVTAIYGQPKPGYSGAGFVWMQNSGTLTFDVTVPETGMYTISTRYMQELSTDGRLQYLSVNGINKGSFMLPYTTVWSDFNFGIYKLTKGSNTIQIKAGWGFAYFDTFTVDYAKLDSLNVAPTLTDSQATSETKLLMKYLTEVYGKHIISGQQEIYGGGNNGNSELEFDWIHNLTGKYPAIRGFDFMNYNPLYGWEDGTTNRIIDWVNNKNGIATGCWHITVPKDFNSYKLGDFVDWQKTTYKPTETNFKTANAVIPGTKEYQYVMLTIDDLAKQLLILQQNNVPIILRPYHEAEGNGGANGEGAWFWWASAGAKVYKQLWEMLYTRLTQTYGLHNIIWTYNSYVYSTSPAWYPGDNLVDIVGYDKYNTVYNRYDGLSGVPNEDAITTIFYQLVDLTNGKKMVAMTENDTVPSLQNLTEEKSGWLYFCPWYGEYLMSSANNYTKTLKELYQSNYVITLDELPNLKVDDQTPSSTITPTSITFDKYTPNQSDKTISIVFNGNTLNSLKLGTTTLKINVDYTLSGNNLILKKTFLSQLPTGESSIVFDFDKGKDPVLKVNVVDSTPSATITPVNATFDKAVDKQQNINVTVNLNGLSLVSVLNNSNKLLLNKDYTVSGNTIVLQKSYLSTLSLGESKLTFQFSAGNNPVLTLNVIDTTAPVITGNLVIQAFNGNTSASTNGISPKFKLINTGNTDINLSDIKLRYYYTIDGEISQNFWCDWASVGSENVKGEFVKLAKPVSGADYYLEISFTASAGTLKAGQNAEIQARFAKSNWSNYNQADDYSFNASSSQYVNSDKVTGYLSNKLVWGIEP

SEQ ID NO: 28 - protein, 541 aa, Clostridium phytofermentans
MAKDIKYSADARVAMEAGVNKLANTVRVTLGPKGRNVVLDKSFGAPLITNDGVTIAKEIELEDSFENMGAQLVKEVATKTNDVAGDGTTTATVLAQAMINEGMKNLAAGANPIILRRGMKKATDCAVEAISSMSSAINGKDQIAKVAAISAGDDSVGEMVADAMDKVSKDGVITIEESKTMQTELDLVEGMQFDRGYVSAYMATDMDKMEANLDNPYILITDKKISNIQEILPVLEQIVQSGSRLLIIAEDIEGEALTTLVINKLRGTFTVVGVKAPGYGDRRKAMLQDIAILTGGTVISDELGLDLKEATLDQLGRAKSVKIQKENTIIVDGEGNKAEIEARISQIKAQIAETTSEFDKEKLQERLAKLAGGVAVIRVGAATETEMKEKKLRMEDALAATRAAVEEGIIAGGGSAYIHASKEVAKLAAKLEGDERTGAQIILKALEAPLSCIAQNAGLEGAVIVNKVREKKTGVGFNALTEKYVDMVEDGILDPSKVTRSALQNATSVASTFLTTEAAVASIKEPAPAMPAGGPGGMGMM

SEQ ID NO: 29 - protein, 95 aa, Clostridium phytofermentans
MKLVPLGDKVVLKQLVAEETTKSGIVLPGQAKEKPQQAEVVAVGPGGMVDGKEVTMQVKVGDKVIYSKYAGTEVKLDEEEFIVVKQNDIVAIVAD

SEQ ID NO: 30 - DNA, 24 bp, Clostridium phytofermentans
cactttctattgccagtatggtac

SEQ ID NO: 31 - DNA, 33 bp, Artificial Sequence
  Description of Artificial Sequence Synthetic oligonucleotide
gtggtggtggtggtggtgttttccataatattg

SEQ ID NO: 32 - DNA, 425 bp, Clostridium phytofermentans
agacagcataagtcacatccagacaaatgtcctataggatgttagtaggggtttggagaattgcccgtaaggcaggttatttggctagatataatcaatccagttacaggatagtaggattgcaacccagtcgttttgaccagtttgtacaagaattttaatttgtcgaaatattgtggcaaatcaaatgaagttctttgatgaaatgtttagaaacatgacttagaatggggtacaaaaagtgaatttgtaagcaaaaagacttgacctttcctacgatagttgttataatcatcttgttattggaacgattatatttacttatgcacattttagagtttttcgaattgttaatacatcattaacaatttaattatactcgttatgtgacgtaagtcaatataatacaaaaccatatattttaa